(19) United States
(12) Patent Application Publication
(10) Pub. No.: US 2009/0136566 A1
(43) Pub. Date: May 28, 2009

(54) THERAPEUTIC TRITERPENOIDS

(75) Inventors: Pavel A. Krasutsky, Duluth, MN (US); Igor V. Kolomitsyn, Duluth, MN (US); Jon M. Holy, Duluth, MN (US); Edward Leon Perkins, Duluth, MN (US); Oksana Kolomitsyna, Duluth, MN (US)

Correspondence Address: SCHWEGMAN, LUNDBERG & WOESSNER, P.A., P.O. BOX 2938, MINNEAPOLIS, MN, 55402, US

(73) Assignee: Regents of the University of Minnesota, St. Paul, MN (US)

(21) Appl. No.: 12/250,401

(22) Filed: Oct. 13, 2008

Related U.S. Application Data
(63) Continuation of application No. PCT/US2007/066632, filed on Apr. 13, 2007.
(60) Provisional application No. 60/792,097, filed on Apr. 13, 2006.

(51) Int. Cl. A61K 9/127; A61K 31/565; A61K 8/63; C07J 75/00; A61P 35/00

(52) U.S. Cl. 424/450; 514/170; 514/171; 424/59; 552/514

(57) ABSTRACT

The present invention relates generally to compositions that can be obtained by extraction of birch bark, methods of using such compositions (e.g., methods of medical use, cosmetic use and/or pharmaceutical use), food products and methods of manufacturing such compounds. The compositions are triterpenes, triterpene alcohols, or derivatives of triterpene alcohols.

### RELATED APPLICATIONS

This application is a continuation under 35 U.S.C. 111 (a) of International Application No. PCT/US2007/066632, filed Apr. 13, 2007 and published as WO 2007/121352 on Oct. 25, 2007, which claims priority from U.S. Provisional Application No. 60/792,097, filed Apr. 13, 2006, which applications are incorporated herein by reference in their entirety.

### FIELD OF THE INVENTION

The field of the present invention is the use of certain triterpene derivatives including esters, which can be extracted from birch bark or prepared by derivatization of birch bark constituents, as dietary supplements, cosmetic ingredients, antibiotics such as anti-bacterials, anti-fungals, anti-protozoans and anti-parasitics, and in the prevention and treatment of cancer.

### BACKGROUND OF THE INVENTION

Birch bark is at the present time a low value product in the forest products industry. Eckman, R. (1983), Holzforschung, 37, 205. A single paper mill can generate 70 tons of birch bark per day. Birch bark is a potential source for a variety of organic chemicals; several triterpenoids have been identified in birch bark extracts. For example lupeol, betulin, betulin aldehyde, betulinic acid, methyl betulinate, lupenone, betulonic aldehyde, betulonic acid, β-amyrin, erythrodiol, oleanolic aldehyde, oleanolic acid, methyl oleanolate, and acetyl oleanolic acid are all present in the bark of Betula verrucosa. Eckerman, C. (1985), Paperi ja Puu, 3, 100.

Some of these components have been shown to have useful pharmacologic properties; for instance, the anti-viral activity of betulin, such as against herpesvirus, has been demonstrated (U.S. Pat. No. 5,750,578). Betulin has also been shown to possess anti-inflammatory activity (Recio, M. (1995), Planta Med., 61, 5). Betulinic acid has been shown to have antitumor activity against human melanoma, (Pisha, E., et al. (1995), J. M. Nature Medicine, 1, 1046) and anti-HIV activity (Fujioka, T., et al. (1994), J. Nat. Prod., 57, 243). Lupeol caffeate has been shown to have anti-malarial activity (Chumkaew, P., et al. (2005), Chem. Pharm. Bull. 53(1), 95-96.

There is an ongoing need for new and improved bioactive agents active against a wide variety of malconditions, such as viral infections, bacterial and fungal infections, cancer, and others. There is also a need for compositions useful for skin health (cosmetic ingredients) and for use as dietary supplements to improve overall health. Lastly, there is a need to utilize the abundant natural resource of birch bark in productive ways.

### SUMMARY OF THE INVENTION

Embodiments of the present invention concern compositions comprising triterpene derivatives, including esters, such as unsaturated aralkenoyl esters. Further embodiments are directed to methods of using these compositions in the treatment of hyperproliferative diseases such as cancer, as dietary supplements, and as cosmetic ingredients such as UV screens. Other embodiments are directed to methods of using these compositions as antibiotics. Some of the compositions of the invention can be obtained by the extraction of birch bark. Other inventive compositions can be derived from the chemical derivatization of natural product birch bark constituents and their structural analogs. Methods of semi-synthesis of these compounds are also provided.

The present invention is directed to a composition that includes at least two of: (a) betulin 3-caffeate; (b) betulinic acid; (c) oleanolic acid; (d) betulin; (e) lupeol; (f) 3-acetoxyoleanolic acid; (g) betulin aldehyde; (h) betulonic aldehyde; and (i) pycarehic acid (betulinic acid-3-caffeate); wherein the composition is essentially free of plant tissue.

The present invention also is directed to a composition that includes: (a) betulin 3-caffeate; (b) betulinic acid; (c) oleanolic acid; (d) betulin; (e) lupeol; (f) 3-acetoxyoleanolic acid; (g) betulin aldehyde; (h) betulonic aldehyde; and (i) pycarehic acid (betulinic acid 3-caffeate); wherein the composition is essentially free of plant tissue.

The present invention also is directed to a composition that includes: (a) up to about 10.0 wt. % of betulin 3-caffeate; (b) up to about 20.0 wt % of betulinic acid; (c) up to about 10.0 wt. % of oleanolic acid; (d) up to about 80.0 wt. % of betulin; (e) up to about 15.0 wt. % of lupeol; (f) up to about 15.0 wt. % of 3-acetoxyoleanolic acid; (g) up to about 1.5 wt. % of betulin aldehyde; (h) up to about 1.0 wt. % of betulonic aldehyde; and (i) up to about 10.0 of pycarehic acid (betulinic acid 3-caffeate); wherein the composition is essentially free of plant tissue. In the inventive compositions, when present in the composition, the disclosed compounds are present in any suitable and effective amount.

An embodiment of the invention also provides a method of treating a hyperproliferative disease in a mammal, the method includes administering to the mammal in need of such treatment an effective amount of any of the above-described compositions in a dosage, at a frequency, and for a duration of time sufficient to provide a beneficial result.

An embodiment of the invention also provides a method of treating a hyperproliferative disease in a mammal, the method includes administering to the mammal in need of such treatment an effective amount of a compound of formula (I)

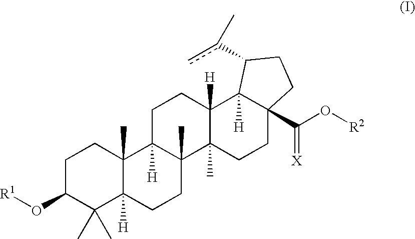

wherein the substituents are as defined herein.

The present invention also provides a method of treating a hyperproliferative disease in a mammal, the method includes administering to the mammal in need of such treatment an effective amount of a compound of formula (II)

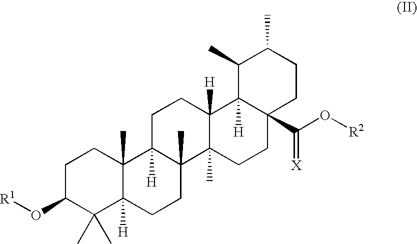

wherein the substituents are as defined herein.

The present invention also provides a method of treating a hyperproliferative disease in a mammal, the method includes administering to the mammal in need of such treatment an effective amount of a compound of formula (III)

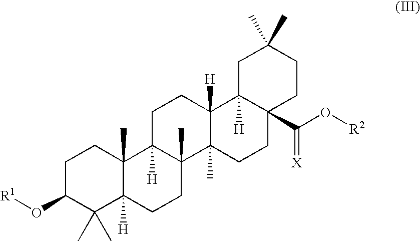

wherein the substituents are as defined herein.

In embodiments of the methods employing the compounds of formulae (I), (II), and (III), R¹ can be a group of formula (IV)

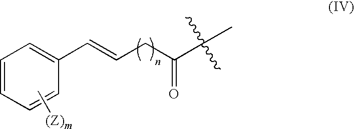

wherein the non-aromatic carbon-carbon double bond is in the cis- or trans-configuration; n is 0-5, m is 0-5; each Z is independently H, OH or hydroxyalkyl; and the wavy line indicates a point of attachment.

An embodiment of the present invention is also directed to a method selected from the group consisting of treating a hyperproliferative disease, providing an antibiotic treatment, providing a dietary supplement, and providing a skin care supplement, in a mammal; the method comprising administering a compound of formula (IVA) in a dosage, at a frequency, for a duration of time, and to a site on or within the mammal, sufficient to treat the mammal;

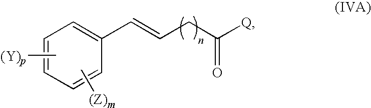

wherein

the non-aromatic carbon-carbon double bond is in the cis- or trans-configuration;

n is 0-5; m is 0-5;

p is 0-5, provided that m+p is less than or equal to a total of 5;

each Y is independently alkyl, alkenyl, alkoxy, halo, haloalkyl, hydroxy, hydroxyalkyl, aryl, heteroaryl, heterocycle, cycloalkyl, alkanoyl, acyloxy, alkoxycarbonyl, acylamino, nitro, trifluoromethyl, trifluoromethoxy, carboxy, carboxyalkyl, alkylthio, alkylsulfinyl, alkylsulfonyl, cyano, acetamido, acetoxy, acetyl, arylamido, arylsulfinyl, arylsulfonamido, arylsulfonyl, arylsulfonylamino, aroyl, arylamino, aroyloxy, aralkyl, aralkyloxy, aralkyloxycarbonyl, aralkylthio, carbamoyl, carbamate, isocyanato, sulfamoyl, sulfinamoyl, sulfino, sulfo, sulfoamino, thiosulfo, NR^{x}R^{y} or COOR^{x}, wherein each R^{x} and R^{y} is independently at each occurrence H, or substituted or unsubstituted alkyl, alkenyl, aryl, heteroaryl, heterocyclyl, or cycloalkyl; each Z is independently H, OH or hydroxyalkyl; and

Q comprises a residue of betulin, betulinic acid, ursolic acid, oleanic acid, allobetulin, allobetulin lactone, lupeol, or a pentacyclic triterpene alcohol; bonded by a hydroxyl thereof to the carbonyl group.

The present invention also provides a method of providing topical UV-protection to a mammal, the method includes topically applying the composition of the present invention to the mammal before the mammal is exposed to UV radiation.

The present invention also provides a method of treating cancer associated with UV radiation, the method includes topically applying the composition of the present invention to the mammal before the mammal is exposed to UV radiation.

The present invention also provides a method of treating a fungal or bacterial infection by use of a composition of the invention at a dosage, with a frequency and for a duration effective to provide a beneficial effect to a mammal in need thereof.

The present invention also provides a method of preparing a compound of formula (V):

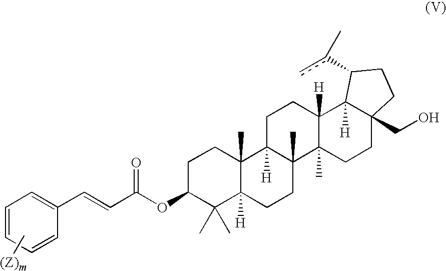

wherein the bond represented by [symbol] is absent or present, and each Z is independently hydrogen, hydroxyl, alkoxyl, hydroxyalkyl, halo, alkyl, aryl, aralkyl, or acyloxy; and m=0-5; the method including contacting a compound of formula (VI):

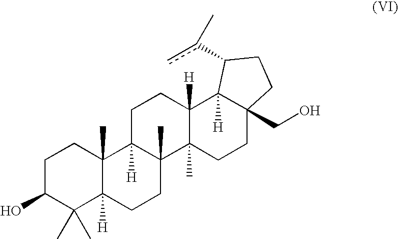

and at least two molar equivalents of an α-haloacetyl halide or an α-haloacetic anhydride in a first organic solvent to provide a compound of formula (VII):

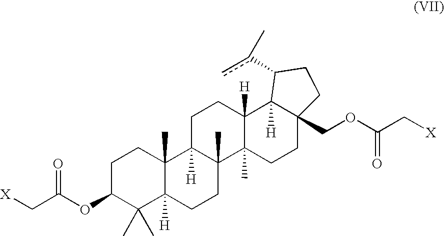

wherein X is chloro, bromo, or iodo; then, contacting the compound of formula (VII) and an aluminum alkoxide in a second organic solvent under conditions of sufficient temperature and time to provide a compound of formula (VIII):

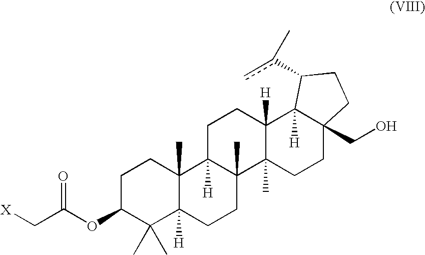

then, contacting the compound of formula (VIII) and a triarylphosphine under conditions of sufficient temperature and time to provide a compound of formula (IX):

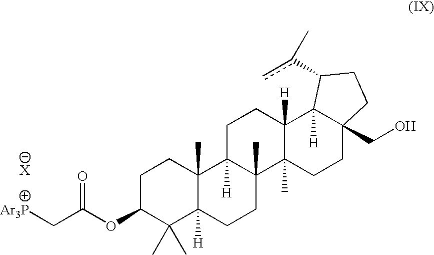

wherein Ar comprises an aryl or heteroaryl and X⁻ is a halide; and then, contacting the compound of formula (IX) and a benzaldehyde, the benzaldehyde being optionally substituted with about 1 to 5 substituents from the group consisting of hydrogen, hydroxyl, alkoxyl, hydroxyalkyl, halo, alkyl, aryl, aralkyl and acyloxy; in the presence of base, under conditions of sufficient temperature and time, to provide the compound of formula (V).

The present invention also provides a method of preparing betulin 3-caffeate, including:

contacting betulin and at least two molar equivalents of a α-haloacetyl halide in a first organic solvent under conditions of sufficient temperature and time to provide a 3-O,28-O-bis(α-haloacetyl)-betulin;

contacting the 3-O,28-O-bis(α-haloacetyl)-betulin and an aluminum alkoxide in a second organic solvent under conditions of sufficient temperature and time to provide a 3-O-(α-haloacetyl)-betulin;

contacting the 3-O-(α-haloacetyl)-betulin and a triarylphosphine under conditions of sufficient temperature and time to provide a 3-O-(α-triarylphosphoniumacetyl)-betulin salt; and

contacting the 3-O-(α-triarylphosphoniumacetyl)-betulin salt and 3,4-dihydroxybenzaldehyde in the presence of base under conditions of sufficient temperature and time to provide betulin 3-caffeate.

The present invention also provides a method of preparing a compound of formula (XV):

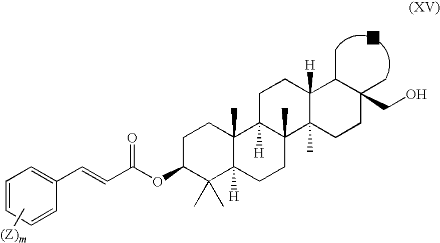

wherein A comprises a segment forming, together with the atoms to which it is attached, a 5- or 6-membered ring bearing alkyl or alkenyl substituents, and each Z is independently hydrogen, hydroxyl, alkoxyl, hydroxyalkyl, halo, alkyl, aryl, aralkyl, or acyloxy, and m=0-5; the method comprising:

contacting a compound of formula (XVI):

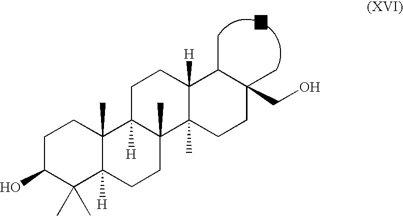

and at least two molar equivalents of an α-haloacetyl halide or an α-haloacetic anhydride in a first organic solvent to provide a compound of formula (XVII):

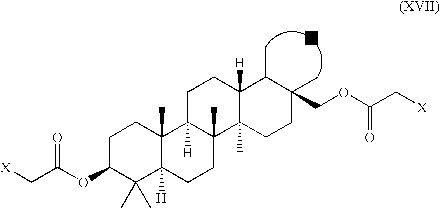

wherein X is chloro, bromo, or iodo; then,

contacting the compound of formula (XVII) and an aluminum alkoxide in a second organic solvent under conditions of sufficient temperature and time to provide a compound of formula (XVIII):

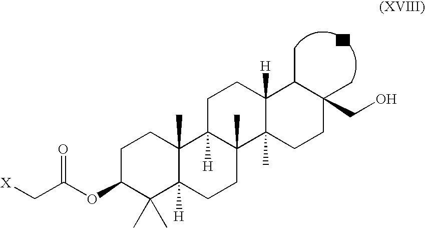

then, contacting the compound of formula (XVIII) and a triarylphosphine under conditions of sufficient temperature and time to provide a compound of Formula (XIX):

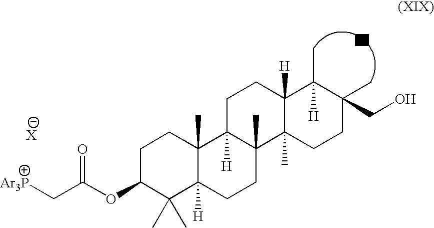

wherein Ar comprises an aryl or heteroaryl group, and X⁻ is halide; and then contacting the compound of Formula (XIX) and a benzaldehyde, the benzaldehyde being optionally substituted with about 1 to 5 substituents from the group consisting of hydrogen, hydroxyl, alkoxyl, hydroxyalkyl, halo, alkyl, aryl, aralkyl and acyloxy; in the presence of base, under conditions of sufficient temperature and time, to provide the compound of formula (XV).

The present invention also provides a method of preparing a compound of formula (XXV):

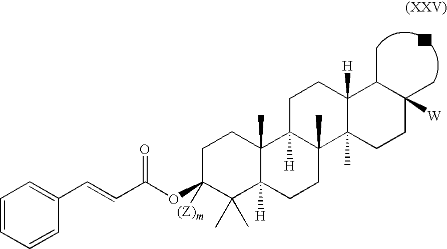

wherein A comprises a segment forming, together with the atoms to which it is attached, a 5- or 6-membered ring bearing alkyl or alkenyl substituents, W is H, alkyl, ether, carboxy, alkylcarboxy, cycloalkyl, or aryl, or W together with a segment of the ring comprising A form a cyclic group that can comprise a heteroatom; and each Z is independently hydrogen, hydroxyl, alkoxyl, hydroxyalkyl, halo, alkyl, aryl, aralkyl, or acyloxy, and m=0-5; the method comprising:

contacting a compound of formula (XXVI):

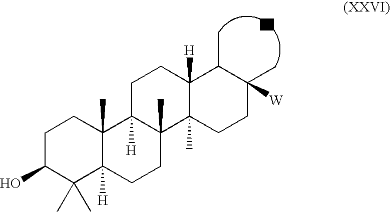

and at least one molar equivalent of an α-haloacetyl halide or an α-haloacetic anhydride in a first organic solvent to provide a compound of formula (XXVII):

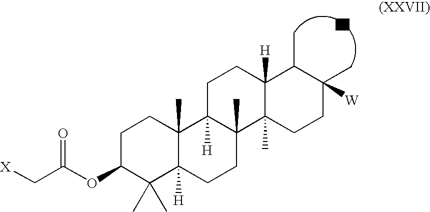

wherein X is chloro, bromo, or iodo; then,

contacting the compound of formula (XXVII) and a triarylphosphine under conditions of sufficient temperature and time to provide a compound of Formula (XXIX):

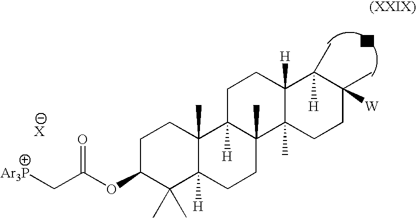

wherein Ar comprises an aryl or heteroaryl group, and X⁻ is halide; and then

contacting the compound of Formula (XIX) and a benzaldehyde, the benzaldehyde being optionally substituted with about 1 to 5 substituents from the group consisting of hydrogen, hydroxyl, alkoxyl, hydroxyalkyl, halo, alkyl, aryl, aralkyl and acyloxy; in the presence of base, under conditions of sufficient temperature and time, to provide the compound of formula (XXV).

The present invention also provides a method of preparing a compound of formula (X):

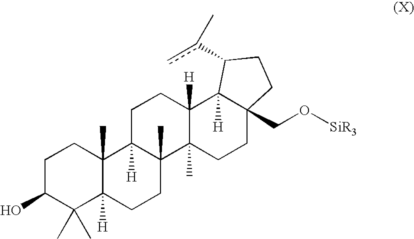

wherein the bond represented by [symbol] is absent or present and each R is independently alkyl or aryl;

the method including: contacting a compound of formula (VI):

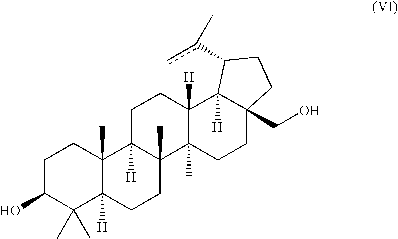

wherein the bond represented by [symbol] is absent or present, and a silyl derivative comprising an R₃Si group wherein R is independently at each occurrence alkyl or aryl or any combination thereof, in an organic solvent and a base, to provide the compound of formula (X).

The present invention also provides a method of preparing a compound of formula (X):

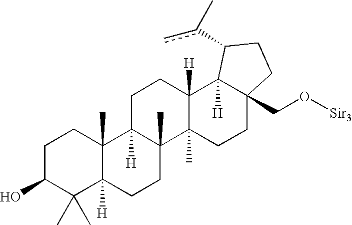

wherein the bond represented by [symbol] is absent or present and each R is independently alkyl or aryl;

the method includes: contacting, at a temperature of about 50° C. to about 70° C. for about 12 to about 48 hours, a compound of formula (VI):

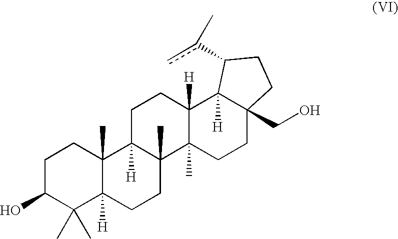

4-(N,N-dimethylamino)-pyridine, at least a 5.0 molar excess of tert-butyldiphenylsilylchloride relative to the compound of formula (VI), triethylamine and chloroform, to provide the compound of formula (X).

### BRIEF DESCRIPTION OF THE DRAWINGS

FIG. 1 is a bar graph showing the percent inhibition of P19 stem cell growth by different extracts and purified components of Betula species. For a key to the sample identities, see Table 2 in the Examples. Samples 1-8 represent different extracts (samples 2-8), along with a mixture of suberinic and betulinic acids (sample 1). Samples 9-13 include docosanedioic acid and related compounds, and samples 14-18 include various lupane-type compounds.

FIG. 2 is a graph showing the results of dose-response studies comparing the effectiveness of extracts from four species of birch (B. papyrifera, B. kenaica, B. neoalaskana, and B. pendula) with betulinic acid (BetA) and betulin 3-caffeate (Bet3C). Concentrations of from 2.5 to 20 μg/ml were added to cultures of P19 stem cells for 48 h, and the numbers of surviving cells measured using sulforhodamine B assays. This graph shows the average of four independent experiments (error bars omitted for clarity).

FIG. 3 is a bar graph showing a comparison of the effects of betulin 3-caffeate on other types of malignant cancer cell lines. P19 stem cells, K1735-M2 melanoma cells and MCF-7 breast cancer cells were treated with 2.5-10 mg/ml betulinic acid or betulin-3-caffeate for 48 h and cell quantity determined by sulforhodamine B assays.

FIG. 4 is a bar graph showing measurements of P19 cell death induced by extracts, betulinic acid (BetA) and betulin 3-caffeate (Bet3C). Because the reduction in cell numbers detected by sulforhodamine B assays could result from either an inhibition of cell proliferation or an induction of cell death (or a combination of both), propidium iodide labeling was used in conjunction with flow cytometry to detect dead and dying cells with compromised plasma membranes.

FIG. 5 shows the UV spectra of extracts of Betulin 3-caffeate.

FIG. 6 shows the UV spectra of extracts of Betula papyrifera and Betula verrucosa. The UV spectrum was recorded as a methanol solution of extracts of Betula papyrifera (American paper birch) and Betula verrucosa (European paper birch). The concentration of extracts were C_{Betula papyrifera}=0.23 mg/ml; C_{Betula verrucosa}=0.29 mg/ml.

### DETAILED DESCRIPTION OF THE INVENTION

Reference will now be made in detail to certain claims of the invention, examples of which are illustrated in the accompanying structures and formulas. While the invention will be described in conjunction with the enumerated claims, it will be understood that they are not intended to limit the invention to those claims. On the contrary, the invention is intended to cover all alternatives, modifications, and equivalents, which may be included within the scope of the present invention as defined by the claims.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention relates to compositions, methods of using such compositions (e.g., methods of medical use, cosmetic use and/or pharmaceutical use), food products and methods of manufacturing compounds. When describing the compositions, methods of using such compositions, food products and methods of manufacturing the compounds, the following terms have the following meanings, unless otherwise indicated.

### DEFINITIONS

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

As used herein, “pharmaceutically acceptable salts” refer to compounds described herein, wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds described herein can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., (1985), 1418 the disclosure of which is hereby incorporated by reference.

The term “stereoisomers” refers to enantiomers, diastereomers, or any other form of spatial isomerism as are well-known in the art. Any depiction of molecular structure herein, unless a stereochemical configuration is depicted, for example by using solid and dashed wedges as is well-known in the art, is taken to include all possible stereochemical configurations of the depicted structure. Examples are R and S configurations at any chiral center, D and L, or d and l, designations of a given molecule, and the like. It is understood that one diastereomer of a compound disclosed herein may display superior activity compared with the other. When required, separation of stereochemically mixed material can be achieved, for example by using HPLC using a chiral column or by a resolution using a resolving agent such as camphonic chloride as in Tucker et al., J. Med. Chem., 37:2437 (1994) to separate racemic mixtures of enantiomers, or by HPLC, column chromatography, crystallization, and the like to separate diastereomeric mixtures. A chiral compound, or a particular diastereotopic chiral center may also be directly synthesized using a chiral catalyst or a chiral ligand, e.g. Huffman et al., J. Org. Chem., 60:1590 (1995).

“Therapeutically effective amount” is intended to include an amount of a compound described herein, or an amount of the combination of compounds described herein, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul., 22:27 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or (iv) diminishing symptoms associated with the pathologic condition.

“Antibiotic” or “antibiotic activity” refers to antibacterial, antifungal, anti-protozoan (e.g., malaria, Guiardia), and anti-parasitic (anti-helmitic) biological activity.

“Stable compound” and “stable structure” are meant to indicate a compound that is sufficiently robust to survive isolation to a useful degree of purity from a reaction mixture, and formulation into an efficacious therapeutic agent. Only stable compounds are contemplated herein.

As used herein, a “residue of a compound” is a radical of a compound of the given structure having one or more open valences. Any synthetically feasible atom or atoms of the compound may be removed to provide the open valence. Based on the linkage that is desired, one skilled in the art can select suitably functionalized starting materials that can be derived from a compound using procedures that are known in the art. For example, suitable atoms that may be removed include a hydrogen atom from the OH group of the triterpenoid alcohol, for example betulin, providing a betulin radical that can be bonded, for example with another residue including a carbonyl group, to provide an ester of betulin.

“Substituted” is intended to indicate that one or more hydrogen atoms bonded to the atom indicated in the expression using “substituted” is replaced with a selection from the indicated group(s), provided that the indicated atom's normal valency is not exceeded, and that the substitution results in a stable compound. Suitable indicated substituent groups include, e.g., alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, acyloxy, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyanato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy. When a substituent is keto or oxo (i.e., ═O) or thioxo (i.e., ═S) group, then 2 hydrogens on the atom are replaced.

Specific and preferred values listed below for radicals, substituents, and ranges, are for illustration only; they do not exclude other defined values or other values within defined ranges for the radicals and substituents.

“Alkyl” refers to a C₁-C₁₈ hydrocarbon containing normal, secondary, tertiary or cyclic carbon atoms. Examples are methyl (Me, —CH₃), ethyl (Et, —CH₂CH₃), 1-propyl (n—Pr, n-propyl, —CH₂CH₂CH₃), 2-propyl (i—Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃. The alkyl can be a monovalent hydrocarbon radical, as described and exemplified above, or it can be a divalent hydrocarbon radical (i.e., alkylene).

The alkyl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyanato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy. The alkyl can optionally be interrupted with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO) or sulfonyl (SO₂). Additionally, the alkyl can optionally be at least partially unsaturated, thereby providing an alkenyl.

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyanato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyanato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyanato, sulfamoyl, sulfinamoyl, sulfino, sulfo, sulfoamino, thiosulfo, NR^{x}R^{y} and/or COOR^{x}, wherein each Rx and R^{y} are independently H, alkyl, alkenyl, aryl, heteroaryl, heterocycle, cycloalkyl or hydroxy.

The cycloalkyl can optionally be at least partially unsaturated, thereby providing a cycloalkenyl.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidazolyl, indazolyl, indolizinyl, indolyl, isobenzofuranyl, isoindolyl, isoquinolyl, isothiazolyl, isoxazolyl, naphthyridinyl, naphtho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyanato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

The term “heterocycle” or “heterocyclyl” refers to a saturated or partially unsaturated ring system, containing at least one heteroatom selected from the group oxygen, nitrogen, and sulfur (which can bear additional oxygen atoms, as in a sulfoxide or sulfone, or nitrogen atoms, as in a sulfoximine), and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyanato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. In one specific embodiment of the invention, the nitrogen heterocycle can be 3-methyl-5,6-dihydro-4H-pyrazino[3,2,1-jk]carbazol-3-ium iodide.

The term “alkanoyl” refers to C(═O)R, wherein R is an alkyl group as previously defined. An “aroyl” or heteroaroyl group refers to C(═O)R wherein R is an aryl or heteroaryl group respectively.

The term “acyloxy” refers to —O—C(═O)R, wherein R is an alkyl, alkyl, cycloalkyl, aryl, or heteroaryl group as previously defined. Examples of acyloxy groups include, but are not limited to, acetoxy, propanoyloxy, butanoyloxy, benzoyloxy, and pentanoyloxy.

The term “alkoxycarbonyl” refers to C(═O)OR, wherein R is an alkyl group as previously defined. Examples of an alkoxycarbonyl group include a t-butoxycarbonyl group (t-Boc) or a benzyloxycarbonyl group (Cbz).

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl, cycloalkyl, aryl, or heteroaryl.

The term “imino” refers to —C(═NH)—. The imino can optionally be substituted with one or more alkyl, alkenyl, alkoxy, aryl, heteroaryl, heterocycle or cycloalkyl.

The term “carboxy” refers to —C(═O)OH. The term “carbonyl” refers to —C(═O)—.

As to any of the above groups, which contain one or more substituents, it is understood, of course, that such groups do not contain any substitution or substitution patterns which are sterically impractical, chemically unstable, and/or synthetically non-feasible. In addition, the compounds of this invention include all stereochemical isomers arising from the substitution of these compounds.

As used herein, “contacting” refers to the act of touching, making contact, mixing, stirring, adding, or of immediate proximity.

As used herein, “separating” refers to the process of removing solids from a mixture. The process can employ any technique known to those of skill in the art, e.g., decanting the mixture, filtering the solids from the mixture, or a combination thereof.

As used herein, “alkaline metal” includes any of the mono-valent metals of group I of the periodic table (e.g., lithium, sodium, or potassium). The hydroxides of the alkali metals are strongly alkaline (basic).

As used herein, “polar solvent” includes solvents that exhibit polar forces on solutes, due to high dipole moment, wide separation of charges, or tight association; e.g., water, alcohols, and acids.

As used herein, “triterpene” or “triterpenoid” refers to a plant secondary metabolite that includes a hydrocarbon, or its oxygenated analog, that is derived from squalene by a sequence of straightforward cyclizations, functionalizations, and sometimes rearrangement. Triterpenes or analogues thereof can be prepared by methods known in the art, i.e., using conventional synthetic techniques or by isolation from plants. Suitable exemplary triterpenes and the biological synthesis of the same are disclosed, e.g., in R. B. Herbert, The Biosynthesis of Secondary Plant Metabolites, 2nd. ed., Chapman, London (1989). The term “triterpene” refers to one of a class of compounds having approximately 30 carbon atoms and synthesized from six isoprene units in plants and other organisms. Triterpenes consist of carbon, hydrogen, and optionally oxygen. Most triterpenes are secondary metabolites in plants. Most, but not all, triterpenes are pentacyclic. Examples of triterpenes include betulin, allobetulin, lupeol, friedelin, and all sterols (most of which are tetracyclic), including lanosterol, stigmasterol, cholesterol, β-sitosterol, and ergosterol.

As used herein, “betulin” refers to 3β,28-dihydroxy-lup-20(29)-ene. Betulin is a pentacyclic triterpenoid derived from the outer bark of paper birch trees (Betula papyrifera, B. pendula, B. verucosa, etc.). The CAS Registry No. is 473-98-3. It can be present at concentrations of up to about 24% of the bark of white birch. Merck Index, 12^{th} Ed., 1236 (1996). Structurally, betulin is shown below:

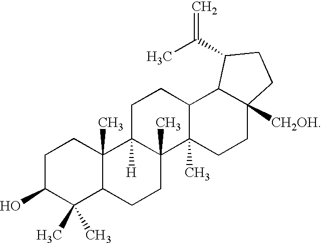

As used herein, “betulinic acid” refers to 3(β)-hydroxy-20(29)-lupaene-28-oic acid; 9-hydroxy-1-isopropenyl-5a,5b,8,8,11a-pentamethyl-eicosahydro-cyclopenta[a]chrysene-3a-carboxylic acid. The CAS Registry No. is 472-15-1. Structurally, betulinic acid is shown below:

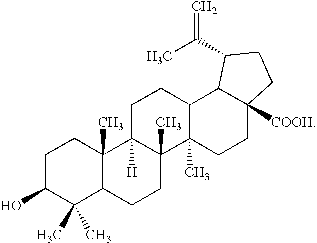

As used herein, “betulin aldehyde” refers to 3(β)-hydroxy-lup-20(29)-en-28-al; Lup-20(29)-en-28-al, 3β-hydroxy-(8CI); Lup-20(30)-en-28-al, 3β-hydroxy-(7CI); 3aH-Cyclopenta[α]chrysene, lup-20(29)-en-28-al deriv.; Betulinaldehyde; Betulinic aldehyde; or Betunal. The CAS Registry Number is 13159-28-9. Structurally, betulin aldehyde is shown below:

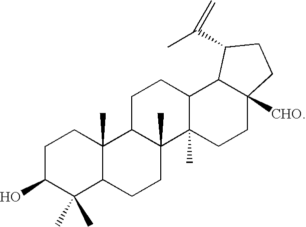

As used herein, “plant material” or “plant tissue” refers to a collection of similar cells of a plant, that typically act together to perform a particular function. The term refers to the tissue of any organism of the plant kingdom, as opposed to one of the animal kingdom or of the kingdoms of Fungi, Protista, or Monera. The plant tissue can be any portion or portions of the plant (e.g., bark, roots, leaves, flowers, needles, bulbs, berries, rhizomes, rootstocks, stems, and seeds), as well as the entire plant. The tissues of a plant (“plant tissue”) generally fall into three main categories: dermal tissue, ground tissue, and vascular tissue. Dermal tissue refers to the “skin” layer of all plant organs and is responsible for environmental interaction (light passage, gas exchange, pathogen recognition and protection, color display, etc.). Dermal tissue is composed of epidermal cells, closely packed cells that secrete a waxy cuticle that aids in the prevention of water loss. Ground tissue lies between dermal tissue and vascular tissue. The ground tissue comprises the bulk of the primary plant body. Parenchyma, collenchyma, and sclerenchyma cells are common in the ground tissue. In roots, the ground tissue may store sugars or starches to fuel the spring sap flow; in leaves, the ground tissue is the layer responsible for photosynthesis (the mesophyll). Vascular tissue transports food, water, hormones and minerals within the plant. Vascular tissue includes xylem, phloem, parenchyma, and cambium cells.

The phrase “essentially free of plant tissue” means that the composition includes less than about 10 wt. % of plant tissue, or less than about 5 wt. % of plant tissue (e.g., plant cells and the like). In some embodiments, the phrase refers to less than about 3 wt. %, less than about 2 wt. %, less than about 1 wt. %, or less than about 0.5 wt. %, of plant tissue.

The term “about” can refer to a variation of +5%, 10%, or 20% of the value specified. For example, “about 50” percent can in some embodiments carry a variation from 45 to 55 percent. For integer ranges, the term “about” can include one or two integers greater than and less than a recited integer.

As used herein, “bark” refers to the dry, dead outer covering of woody branches, stems and roots of plants that is very distinct and separable from the wood itself. It includes all tissue outside the cambium (growth layer between bark and wood).

As used here the terms “leaf” or “leaves” refer to those parts of a plant which grow along the sides of branches or stems or at the bases of plants. Most are green and contain chlorophyll, though they vary in their shapes and sizes. Leaves are the part of the plant that ordinarily performs photosynthesis (the process that converts sunlight and carbon dioxide into energy).

As used herein, “needle” generally refers to a narrow stiff leaf, such as those of conifers (e.g., pine trees).

As used herein, “root” refers to the part of a plant, normally underground, that absorbs nutrients and anchors the plant into the ground.

As used herein, “bulb” refers to a spheroidal body growing from a plant either above or below the ground (usually below), which is usually a bud, consisting of a cluster of partially developed leaves, and producing, as it grows, a stem above, and roots below, (e.g., the onion or tulip bulb). A true bulb is a complete package containing next year's plant (flower) already forming inside. The contents of the bulb are often enclosed in protective, fleshy scales, which are held together by a small basal plate. The scales are modified leaves that contain enough nutrients to sustain the plant through dormancy and early growth. They may be loose and open like those of a lily, or tightly closed like those of a hyacinth. In many bulbs, a paper-thin tunic protects the scales (lilies don't have a tunic). Roots will grow from the bulb's basal plate.

As used herein, “berry” refers to any small fruit that is pulpy or succulent throughout, having seeds loosely imbedded in the pulp, such as the currant, grape, or blueberry. Berry can be further defined as an indehiscent fruit derived from a single ovary and having the whole wall fleshy, such as the grape or tomato. Furthermore, berries come in various structures including simple, such grape; blueberry, cranberry, or aggregate, such as blackberry; raspberry, strawberry mulberry.

As used herein, “rhizome” refers to a horizontal, usually underground stem that often sends out roots and shoots from its nodes (also called rootstalk or rootstock).

As used herein, “rootstock” refers to a robust plant that provides the root system in grafting, also known as a stock. Scions and buds are grafted and budded to a rootstock or stock. Rootstock also refers to the elongated and often thick rhizomes of certain perennial herbaceous plants such as the Iris, Aspidistra and Solomon's Seal.

As used herein, “stem” refers to the main (usually aerial) axis (sometimes referred to as the trunk or stalk) of a tree, shrub, or plant. “Stem” also refers to the part of the plant that supports the leaves, flowers or fruits of a plant, such as the peduncle of a fruit or the pedicel of a flower.

As used herein, “seed” refers to a ripened ovule, consisting of an embryo with one or more integuments, or coverings, such as an apple seed, a currant seed, dill seed, or kola nut seed. By germination, most seeds produce a new plant. “Seed” also refers to any small seedlike fruit, though it may consist of a pericarp, or even a calyx, as well as the seed proper, such as a parsnip seed or thistle seed. The seed proper has an outer and an inner coat, and within these the kernel or nucleus. The kernel is either the embryo alone, or the embryo enclosed in the albumen, which is the material for the nourishment of the developing embryo. The scar on a seed, left where the stem parted from it, is called the hilum, and the closed orifice of the ovule, the micropyle.

A “plant” can be a bryophyte or vascular plant. More specifically, the plant can be grass, flower or a tree and the plant tissue can be any part of the grass, flower or tree. A specific plant is the birch tree, wherein the suitable plant tissue for extracting a composition of the invention can be the bark of the birch tree. As used herein, “birch” or “birch tree” refers to any of the several deciduous tress of the genus Betula. The birches comprise the family Betulaceae in the order Fagales. Birch trees include, for example, white birch, B. alba; sweet, black or cherry birch, B. lenta; monarch birch, B. maximowicziana; dwarf or arctic birch, B. nana; Japanese white birch, B. platphyla japonica; Alaskan birch, B. neoalaskana; Kenai birch, B. kenaica; smooth-bark birch, B. pubescens; yellow birch, B. alleghaniensis; paper, white or canoe birch, B. papyrifera; gray birch, B. populifolia; river birch, B. nigra; and the European birches, B. pubescens; B. alba and B. pendula. Specifically, birch can be B. alba, B. neoalaskana, B. kenaica, B. lenta, B. maximowicziana, B. nana, B. platyphyla japonica, B. pubescens, B. alleghaniensis, B. papyrifera, B. populifolia, B. nigra or B. pendula. A specific birch for use in the processes of the present invention is B. papyrifera. Another birch is B. neoalaskana.

As used herein, “birch bark” refers to inner birch bark and outer birch bark. Inner birch bark is more dense and granular than outer birch bark, while outer birch bark is more flexible and fibrous than inner birch bark. Outer birch bark is light in color, thin (1-5 mm), tough, and of low water-content relative to inner birch bark. The inner bark is darker in color, thicker (3-10 mm) and non-fibrous relative to the outer bark. The inner bark is the portion of the tree wherein significant water transport occurs (i.e., an area of high water content). Due to the differences in the physical properties of inner birch bark and outer birch bark, fragmentation produces outer birch bark shreds and inner birch bark chunks.

### DETAILED DESCRIPTION

Embodiments of the present invention concern compositions comprising triterpene derivatives, including esters, such as unsaturated aralkyl esters. The inventive compositions can be obtained by the extraction of the plant tissues, such as the bark, of certain plant species, such as birch trees. Certain of the compositions of the invention can be obtained by the extraction from birch bark, which may or may not also involve additional processing steps. Other inventive compositions can be derived from the chemical synthesis of natural product birch bark constituents and their structural analogs. Methods of synthesis of these compounds are also provided.

The present invention is directed to a composition that includes at least two of: (a) betulin 3-caffeate; (b) betulinic acid; (c) oleanolic acid; (d) betulin; (e) lupeol; (f) 3-acetoxyoleanolic acid; (g) betulin aldehyde; (h) betulonic aldehyde; and (i) pycarehic acid (betulinic acid-3-caffeate); wherein the composition is essentially free of plant tissue. For example, the composition can include any 2, 3, 4, 5, 6, 7, 8, or 9 of the aforementioned compounds, in any combination. The inventive composition can be obtained by extraction of birch bark, particularly the bark of certain species of birch trees, such as Betula papyrifera, B. neoalaskana, and B. kenaica. The extraction can be carried out with any suitable organic solvent, for example a halocarbon such as chloroform or dichloromethane, or an oxycarbon such as an alcohol or an ether.

The present invention also is directed to composition that includes: (a) betulin 3-caffeate; (b) betulinic acid; (c) oleanolic acid; (d) betulin; (e) lupeol; (f) 3-acetoxyoleanolic acid; (g) betulin aldehyde; (h) betulonic aldehyde; and (i) pycarehic acid (betulinic acid 3-caffeate); wherein the composition is essentially free of plant tissue. The inventive composition can be obtained by extraction of birch bark, particularly the bark of certain species of birch trees, such as Betula papyrifera, B. neoalaskana, and B. kenaica. The extraction can be carried out with any suitable organic solvent, for example a halocarbon such as chloroform or dichloromethane, or an oxycarbon such as an alcohol or an ether.

The present invention also is directed to a composition that includes: (a) up to about 10.0 wt. % of betulin 3-caffeate; (b) up to about 20.0 wt % of betulinic acid; (c) up to about 10.0 wt. % of oleanolic acid; (d) up to about 80.0 wt. % of betulin; (e) up to about 15.0 wt. % of lupeol; (f) up to about 15.0 wt. % of 3-acetoxyoleanolic acid; (g) up to about 1.5 wt. % of betulin aldehyde; (h) up to about 1.0 wt. % of betulonic aldehyde; and (i) up to about 10.0 of pycarehic acid (betulinic acid 3-caffeate); wherein the composition is essentially free of plant tissue. When constituents are present in composition, they can be present in effective amounts.

Again, the inventive composition can be obtained by extraction of birch bark, particularly the bark of certain species of birch trees, such as Betula papyrifera, B. neoalaskana, and B. kenaica. The extraction can be carried out with any suitable organic solvent, for example a halocarbon such as chloroform or dichloromethane, or an oxycarbon such as an alcohol or an ether.

In all three of the above embodiments, the extraction can be carried out by contacting macerated, shredded, comminuted or pelletized birch bark with the solvent, then filtering to remove insoluble materials and then removing the solvent, for example by distillation or evaporation.

Further embodiments are directed to methods of using these compositions in the treatment of hyperproliferative diseases such as cancer, as antibiotics such as antibacterial and antifungal compounds, as dietary supplements, and as cosmetic ingredients such as UV screens. As discussed below in the Examples, the inventive compositions, such as can be obtained from birch bark extracts, provide valuable materials for the uses disclosed and claimed herein. When the compositions are obtained from birch bark, beneficial economic usage is made of the naturally produced birch bark, which is otherwise typically burned as a waste product from birch tree harvesting, lumber, and pulp making industrial operations.

An embodiment of the invention also provides a method of treating a hyperproliferative disease in a mammal, the method includes administering to the mammal in need of such treatment an effective amount of any of the above-described compositions in a dosage, at a frequency, and for a duration of time sufficient to provide a beneficial result.

An embodiment of the invention also provides a method of treating a hyperproliferative disease in a mammal, the method includes administering to the mammal in need of such treatment an effective amount of a compound of formula (I)

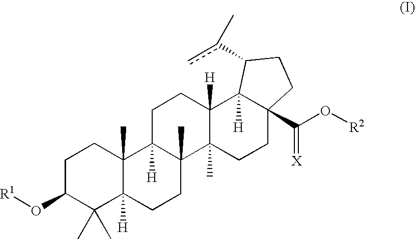

wherein the substituents are as defined herein. R¹ in particular can be a cinnamate ester, i.e., a phenylpropenyl ester, such as a caffeate ester, i.e., a 3,4-dihydroxylphenylpropenoyl ester, or an analog thereof as defined herein. In a preferred embodiment according to the present invention, the compound of formula (I) is 3-O-(caffeoyl)-betulinic acid, wherein R¹ is caffeoyl, R² is H, and X is O.

The present invention also provides a method of treating a hyperproliferative disease in a mammal, the method includes administering to the mammal in need of such treatment an effective amount of a compound of formula (II)

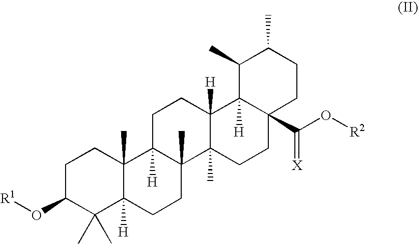

wherein the substituents are as defined herein. R¹ in particular can be a cinnamate ester, i.e., a phenylpropenyl ester, such as a caffeate ester, i.e., a 3,4-dihydroxylphenylpropenoyl ester, or an analog thereof as defined herein. In a preferred embodiment according to the present invention, the compound of formula (I) is 3-O-(caffeoyl)-ursolic acid, wherein R¹ is caffeoyl, R² is H, and X is O.

The present invention also provides a method of treating a hyperproliferative disease in a mammal, the method includes administering to the mammal in need of such treatment an effective amount of a compound of formula (III)

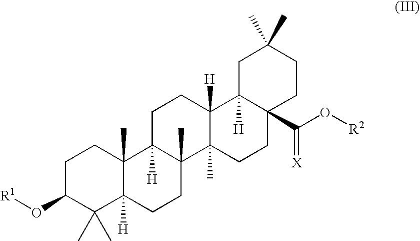

wherein the substituents are as defined herein. R¹ in particular can be a cinnamate ester, i.e., a phenylpropenyl ester, such as a caffeate ester, i.e., a 3,4-dihydroxylphenylpropenoyl ester, or an analog thereof as defined herein. In a preferred embodiment according to the present invention, the compound of formula (I) is 3-O-(caffeoyl)-oleanic acid, wherein R¹ is caffeoyl, R² is H, and X is O.

In embodiments of the methods employing the compounds of formulae (I), (II), and (III), R¹ can be a group of formula (IV)

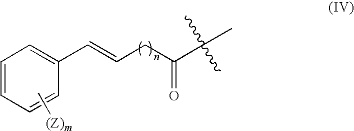

wherein the non-aromatic carbon-carbon double bond is in the cis- or trans-configuration; n is 0-5, m is 0-5; each Z is independently H, OH or hydroxyalkyl; and the wavy line indicates a point of attachment. Thus, in addition to caffeoyl groups, wherein Z is hydroxy, m=2, the position on the ring is 3,4, and n=0, other cinnamate or cinnamate analog groups can be comprised by R¹. A cinnamate analog as the term is used herein includes a structure including a moiety of formula (IV), wherein additional methylene groups can be disposed between the non-aromatic double bond and the carbonyl group, and wherein the ring substitution can be in any of the indicated configurations. By a “non-aromatic double bond” is meant the double bond in the chain, not in the aromatic aryl ring.

Another embodiment of the present invention is directed to a method selected from the group consisting of treating a hyperproliferative disease, providing an antibiotic treatment, providing a dietary supplement, and providing a skin care supplement, in a mammal; the method comprising administering a compound of formula (IVA) in a dosage, at a frequency, for a duration of time, and to a site on or in the mammal, sufficient to treat the mammal;

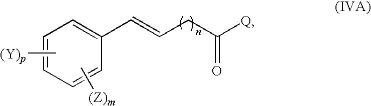

wherein

the non-aromatic carbon-carbon double bond is in the cis- or trans-configuration;

n is 0-5; m is 0-5;

p is 0-5, provided that m+p is less than or equal to a total of 5;

each Y is independently alkyl, alkenyl, alkoxy, halo, haloalkyl, hydroxy, hydroxyalkyl, aryl, heteroaryl, heterocycle, cycloalkyl, alkanoyl, acyloxy, alkoxycarbonyl, acylamino, nitro, trifluoromethyl, trifluoromethoxy, carboxy, carboxyalkyl, alkylthio, alkylsulfinyl, alkylsulfonyl, cyano, acetamido, acetoxy, acetyl, arylamido, arylsulfinyl, arylsulfonamido, arylsulfonyl, arylsulfonylamino, aroyl, arylamino, aroyloxy, aralkyl, aralkyloxy, aralkyloxycarbonyl, aralkylthio, carbamoyl, carbamate, isocyanato, sulfamoyl, sulfinamoyl, sulfino, sulfo, sulfoamino, thiosulfo, NR^{x}R^{y} or COOR^{x}, wherein each R^{x} and R^{y} is independently at each occurrence H, or substituted or unsubstituted alkyl, alkenyl, aryl, heteroaryl, heterocyclyl, or cycloalkyl; each Z is independently H, OH or hydroxyalkyl; and

Q comprises a residue of betulin, betulinic acid, ursolic acid, oleanic acid, allobetulin, allobetulin lactone, lupeol, or a pentacyclic triterpene alcohol; bonded by a hydroxyl thereof to the carbonyl group.

The moiety of the compound of formula (IVA) represented by the entire structure except Q, is a cinnamate or a cinnamate analog within the meaning herein. Therefore according to the definitions herein, a compound of formula (IVA) is a cinnamate or a cinnamate analog derivative of Q. Q can comprise, for example, the residues of the principal triterpene alcohols that can be extracted from birch bark, for example, betulin, betulinic acid and the like, but Q is not limited thereto. Q can also comprise any triterpene alcohol, no matter what the source, from birch, from another species of plant, from another type of living organism, or prepared synthetically.

The present invention also provides a method of providing topical UV-protection to a mammal, the method includes topically applying the composition of the present invention to the mammal before the mammal is exposed to UV radiation. As shown in the Examples, the inventive compositions are effective absorbers of UV radiation, and thus can serve to mitigate the harmful effects of UV light on mammalian skin. UV light is well-known to cause sunburns in humans.

The present invention also provides a method of treating cancer associated with UV radiation, the method includes topically applying the composition of the present invention to the mammal before the mammal is exposed to UV radiation. As shown in the Examples, the inventive compositions are effective absorbers of UV radiation, and thus can serve to mitigate the harmful effects of UV light on mammalian skin. UV light is well-known to cause skin cancer, such as melanomas, in humans.

The present invention also provides a method of treating a fungal or bacterial infection, a protozoan infestation (e.g., malaria, Guiardia), or a parasitic invasion (r.g., Helminthes) by use of a composition of the invention at a dosage, with a frequency and for a duration effective to provide a beneficial effect to a mammal in need thereof. The inventive compositions serve to inhibit the growth of, and to kill, bacterial and fungal cells, and are thus useful in treating, preventing, or palliating infections in mammals such as humans that are caused by such organisms.

### Methods of Manufacturing (Processing)

In the methods of manufacturing described herein, the steps can be carried out in any order without departing from the principles of the invention, except when a temporal or operational sequence is explicitly recited. Recitation in a claim to the effect that first a step is performed, then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The compounds described herein can be prepared by any of the applicable techniques of organic synthesis. Many such techniques are well known in the art. However, many of the known techniques are elaborated in Compendium of Organic Synthetic Methods (John Wiley & Sons, New York) Vol. 1, Ian T. Harrison and Shuyen Harrison (1971); Vol. 2, Ian T. Harrison and Shuyen Harrison (1974); Vol. 3, Louis S. Hegedus and Leroy Wade (1977); Vol. 4, Leroy G. Wade Jr., (1980); Vol. 5, Leroy G. Wade Jr. (1984); and Vol. 6, Michael B. Smith; as well as March, J., Advanced Organic Chemistry, 3rd Edition, John Wiley & Sons, New York (1985); Comprehensive Organic Synthesis. Selectivity, Strategy & Efficiency in Modern Organic Chemistry, In 9 Volumes, Barry M. Trost, Editor-in-Chief, Pergamon Press, New York (1993); Advanced Organic Chemistry, Part B: Reactions and Synthesis, 4th Ed.; Carey and Sundberg; Kluwer Academic/Plenum Publishers: New York (2001); Advanced Organic Chemistry, Reactions, Mechanisms, and Structure, 2nd Edition, March, McGraw Hill (1977); Protecting Groups in Organic Synthesis, 2nd Edition, Greene, T. W., and Wutz, P. G. M., John Wiley & Sons, New York (1991); and Comprehensive Organic Transformations, 2nd Edition, Larock, R. C., John Wiley & Sons, New York (1999).

It is appreciated that those of skill in synthetic organic chemistry understand that reagents are typically referred to by the chemical names that they bear or formulae that represent their structures prior to addition to a chemical reaction mixture, even though the chemical species actually present in the reaction mixture or involved in the reaction may be otherwise. While a compound may undergo conversion to a compound bearing a different name or represented by a different formula prior to or during a specified reaction step, reference to these compounds by their original name or formula is acceptable and is well-understood by those of skill in the art of organic chemistry.

Obviously, numerous modifications and variations of the present invention are possible in light of the above teachings. It is therefore to be understood that within the scope of the appended claims, the invention may be practiced otherwise than as specifically described herein.

The present invention provides a method of preparing a compound of formula (V):

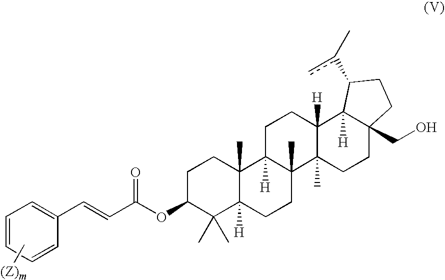

wherein the bond represented by [symbol] is absent or present, and each Z is independently hydrogen, hydroxyl, alkoxyl, hydroxyalkyl, halo, alkyl, aryl, aralkyl, or acyloxy; and m=0-5; the method including contacting a compound of formula (VI):

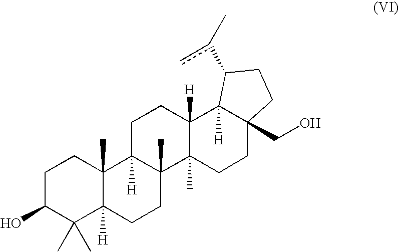

and at least two molar equivalents of an α-haloacetyl halide or an α-haloacetic anhydride in a first organic solvent to provide a compound of formula (VII):

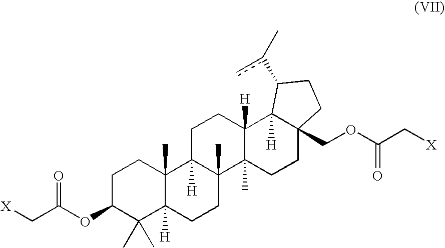

wherein X is chloro, bromo, or iodo; then, contacting the compound of formula (VII) and an aluminum alkoxide in a second organic solvent under conditions of sufficient temperature and time to provide a compound of formula (VIII):

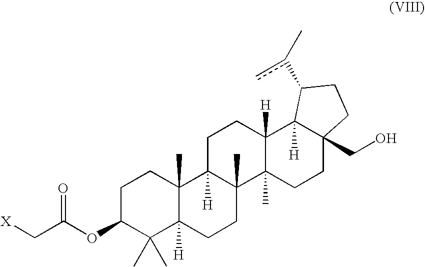

then, contacting the compound of formula (VIII) and a triarylphosphine under conditions of sufficient temperature and time to provide a compound of formula (IX):

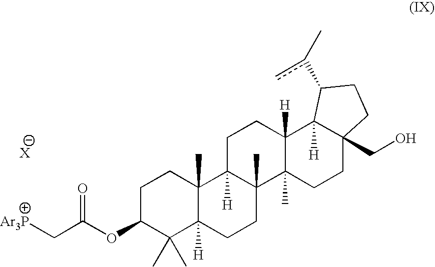

wherein Ar comprises an aryl or heteroaryl and X⁻ is a halide; and then, contacting the compound of formula (IX) and a benzaldehyde, the benzaldehyde being optionally substituted with about 1 to 5 substituents from the group consisting of hydrogen, hydroxyl, alkoxyl, hydroxyalkyl, halo, alkyl, aryl, aralkyl and acyloxy; in the presence of base, under conditions of sufficient temperature and time, to provide the compound of formula (V).

The present invention also provides a method of preparing betulin 3-caffeate, including:

contacting betulin and at least two molar equivalents of a α-haloacetyl halide in a first organic solvent under conditions of sufficient temperature and time to provide a 3-O,28-O-bis(α-haloacetyl)-betulin;

contacting the 3-O,28-O-bis(α-haloacetyl)-betulin and an aluminum alkoxide in a second organic solvent under conditions of sufficient temperature and time to provide a 3-O-(α-haloacetyl)-betulin;

contacting the 3-O-(α-haloacetyl)-betulin and a triarylphosphine under conditions of sufficient temperature and time to provide a 3-O-(α-triarylphosphoniumacetyl)-betulin salt; and

contacting the 3-O-(α-triarylphosphoniumacetyl)-betulin salt and 3,4-dihydroxybenzaldehyde in the presence of base under conditions of sufficient temperature and time to provide betulin 3-caffeate.

The present invention also provides a method of preparing a compound of formula (XV):

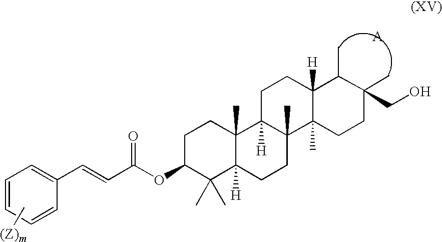

wherein A comprises a segment forming, together with the atoms to which it is attached, a 5- or 6-membered ring bearing alkyl or alkenyl substituents, and each Z is independently hydrogen, hydroxyl, alkoxyl, hydroxyalkyl, halo, alkyl, aryl, aralkyl, or acyloxy, and m=0-5; the method comprising:

contacting a compound of formula (XVI):

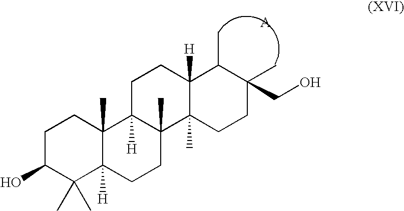

and at least two molar equivalents of an α-haloacetyl halide or an α-haloacetic anhydride in a first organic solvent to provide a compound of formula (XVII):

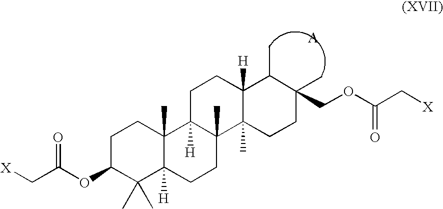

wherein X is chloro, bromo, or iodo; then,

contacting the compound of formula (XVII) and an aluminum alkoxide in a second organic solvent under conditions of sufficient temperature and time to provide a compound of formula (XVIII):

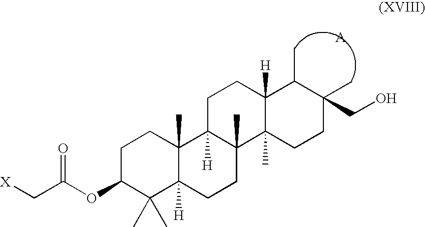

then,

contacting the compound of formula (XVIII) and a triarylphosphine under conditions of sufficient temperature and time to provide a compound of Formula (XIX):

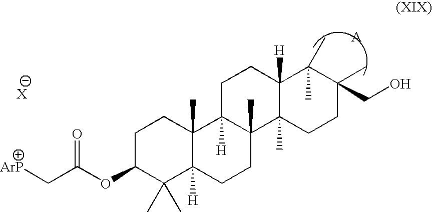

wherein Ar comprises an aryl or heteroaryl group, and X⁻ is halide; and then contacting the compound of Formula (XIX) and a benzaldehyde, the benzaldehyde being optionally substituted with about 1 to 5 substituents from the group consisting of hydrogen, hydroxyl, alkoxyl, hydroxyalkyl, halo, alkyl, aryl, aralkyl and acyloxy; in the presence of base, under conditions of sufficient temperature and time, to provide the compound of formula (XV).

The present invention also provides a method of preparing a compound of formula (XXV):

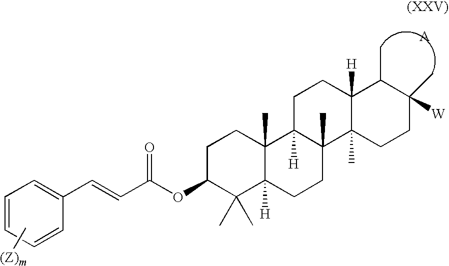

wherein A comprises a segment forming, together with the atoms to which it is attached, a 5- or 6-membered ring bearing alkyl or alkenyl substituents, W is H, alkyl, ether, carboxy, alkylcarboxy, cycloalkyl, or aryl, or W together with a segment of the ring comprising A form a cyclic group that can comprise a heteroatom; and each Z is independently hydrogen, hydroxyl, alkoxyl, hydroxyalkyl, halo, alkyl, aryl, aralkyl, or acyloxy, and m=0-5; the method comprising:

contacting a compound of formula (XXVI):

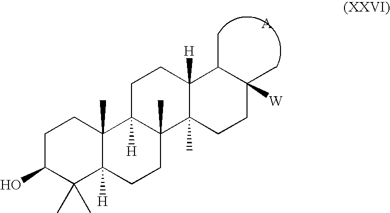

and at least one molar equivalent of an α-haloacetyl halide or an α-haloacetic anhydride in a first organic solvent to provide a compound of formula (XXVII):

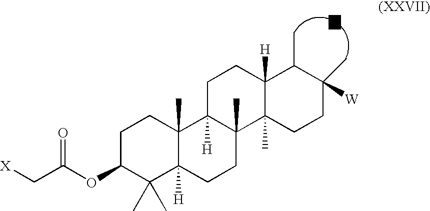

wherein X is chloro, bromo, or iodo; then,

contacting the compound of formula (XXVII) and a triarylphosphine under conditions of sufficient temperature and time to provide a compound of Formula (XXIX):

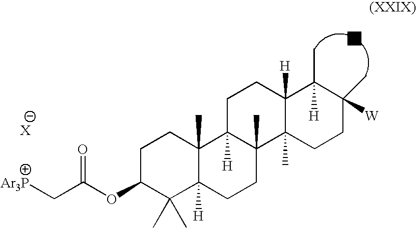

wherein Ar comprises an aryl or heteroaryl group, and X⁻ is halide; and then

contacting the compound of Formula (XIX) and a benzaldehyde, the benzaldehyde being optionally substituted with about 1 to 5 substituents from the group consisting of hydrogen, hydroxyl, alkoxyl, hydroxyalkyl, halo, alkyl, aryl, aralkyl and acyloxy; in the presence of base, under conditions of sufficient temperature and time, to provide the compound of formula (XXV).

The present invention also provides a method of preparing a compound of formula (X):

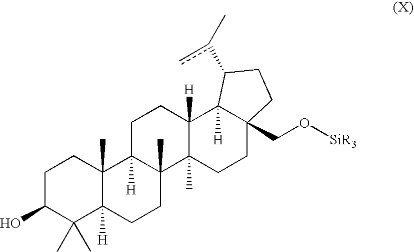

wherein the bond represented by [symbol] is absent or present and each R is independently alkyl or aryl;

the method including: contacting a compound of formula (VI):

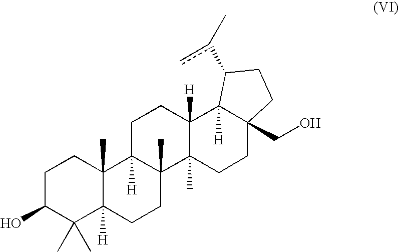

wherein the bond represented by [symbol] is absent or present, and a silyl derivative comprising an R₃Si group wherein R is independently at each occurrence alkyl or aryl or any combination thereof, in an organic solvent and a base, to provide the compound of formula (X).

The present invention also provides a method of preparing a compound of formula (X):

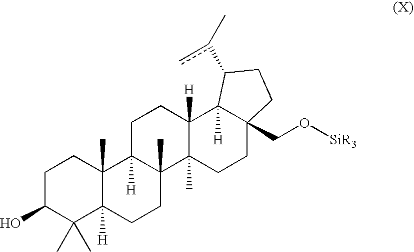

wherein the bond represented by [symbol] is absent or present and each R is independently alkyl or aryl;

the method includes:

contacting, at a temperature of about 50° C. to about 70° C. for about 12 to about 48 hours, a compound of formula (VI):

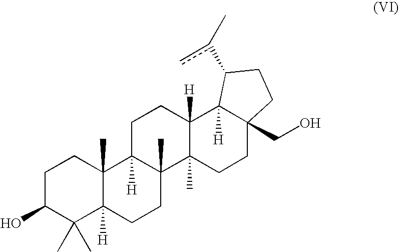

4-(N,N-dimethylamino)-pyridine, at least a 5.0 molar excess of tert-butyldiphenylsilylchloride relative to the compound of formula (VI), triethylamine and chloroform, to provide the compound of formula (X).

### Pharmaceutical Formulations

The compositions of this invention are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients, 5^{th} Ed.; Rowe, Sheskey, and Owen, Eds.; American Pharmacists Association; Pharmaceutical Press: Washington, D.C., 2006. Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences, Mack Publishing Company, Easton, Pa., (1985). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise one or more compounds of the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of a given condition.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

Effective dose of active ingredient depends at least on the nature of the condition being treated, toxicity, whether the compound is being used prophylactically (lower doses), the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

### Routes of Administration

One or more compounds of the invention (herein referred to as the active ingredients) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient.

Specific ranges, values, and embodiments provided below are for illustration purposes only and do not otherwise limit the scope of the invention, as defined by the claims.

### EXAMPLES

The invention will now be illustrated by the following non-limiting Examples. The following examples further define by reference the preparation of the compositions of the invention and their uses. It will be apparent to those skilled in the art that many modifications, both to materials and methods, may be practiced without departing from the purpose and interest of this invention.

### Example 1

### Biological Methods

The following methods were used in several of the examples.

Cell Proliferation Measurements: Sulforhodamine B assays (Skehan, P. et al. (1990). J. Natl. Cancer Inst. 82:1107-1112.) were conducted to measure the effects of compounds and extractives of interest on the proliferation of a number of different cell lines. Cell lines used include mouse P19 and human NT2-D1 stem cells, mouse K1735-M2 melanoma cells, human LNCaP and PC-3 prostate cancer cells, and human MCF-7 breast cancer cells. Other cell lines include normal human fibroblast BJ cells, human Caov-3 ovarian cancer cells, human U87 brain glioma cells, human HL-60 acute promyelocytic leukemia cells, human MOLT-4 acute lymphoblastic leukemia cells, human U937 histiocytic lymphoma cells, human MDA-MB-231 breast cancer cells, C2BBe1 cells (a clone of the human colorectal cell line Caco-2), human K562 chronic myeloid leukemia cells, human WM32-11 primary melanoma cells with radial growth phase-like phenotype (early primary melanoma), and human WM793 melanoma cells with vertical growth phase-like phenotype. Cells were seeded at a concentration of 2.5×10⁴ cells/ml in 24-well plates, and allowed to recover for 2 days prior to drug addition. Test compounds were prepared in most instances as stock solutions in dimethylsulfoxide (DMSO). Compounds were then added to multiwell plates at final concentrations of 0-20 mg/ml. Control wells received an equivalent amount of vehicle (DMSO) only. After 1-3 days of incubation, the culture medium was decanted from each plate, and the cells fixed with cold (−10° C.) absolute methanol containing 1% acetic acid for at least 30 minutes. Subsequently, the methanol was decanted, and the plate air-dried. Sulforhodamine B (0.5% in 1% acetic acid) was added to each well, and the plate incubated at 35° C. for 1 hour. Plates were rinsed with 1% acetic acid, air-dried, and the bound dye eluted with 1 ml of 10 mM Tris buffer, pH 10. The absorbance was measured in a spectrophotometer at 540 nm; the amount of dye released is proportional to the number of cells present in the dish, and is a reliable indicator of cell proliferation.

Morphological examination of experimental and control cell cultures: In addition to proliferation assays, the overall morphological appearance of cells treated with various compounds and extractives was monitored by phase contrast microscopy. Cells seeded and drugged as described above were placed on the stage of an inverted phase contrast microscope and inspected for alterations in cell shape or changes in the proportion of dividing and dead or dying cells.

### Example 2

### UV Radiation Absorption by Betula papyrifera Triterpene Caffeates

Different from other birch bark triterpenes (betulin, betulinic acid, betulinic aldehyde, betulin-3-acetate, betulone, etc.), triterpene caffeates are strong light absorbents. Found below are the results of light absorption analysis (by UV-VIS Spectrophotometer) of compounds 12-15 (Table 1). The range of light absorption (λ) and the level of molar extinction (ε) depicted in Table 1 demonstrate that these chemicals are useful as UV-protectors for sunscreen block materials. Thus, non-purified birch bark extract will provide sun protection if added to, for example, cosmetics.

Sunscreens block the cutaneous absorption of UV radiation at 280-315 nm. This is the same range which is covered by the absorption of triterpene caffeates (see Table 1). The presence of betulin 3-caffeate (or other triterpene derivatives) in cosmetics will prevent sun burning, premature aging and skin cancer (melanoma). The triterpene part of the compound plays a role in such sun-protectors because of their hydrophobic and anti-melanoma nature. Additionally, these ingredients in sun block cosmetics will not be washed away from the skin during, for example, swimming, because of the high hydrophobic action of the triterpene part of the compound.

TABLE 1

| | Light | | ε |
|---|---|---|---|
| | absorption | | (molar |
| | range | | extinction |
| Formula | (λ, nm) | λₘₐₓ | coefficient) |
| 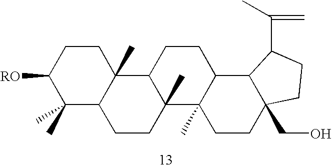 | 200-350 nm | 220 245 305 325 | 13978 10018 13341 15375 |
| 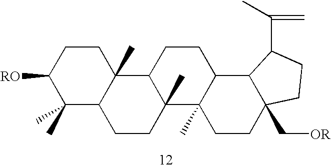 | 210-360 nm | 220 245 305 325 | 2280 1795 2575 2865 |
| 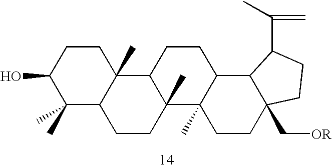 | 210-360 nm | 220 245 305 325 | 1300 875 1190 1380 |
| 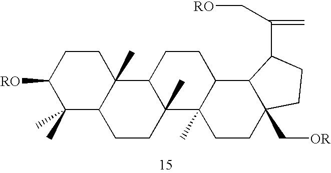 | 210-360 nm | 220 245 305 325 | 1230 882 1174 1353 |

Compound 12 is betulin-3,28-dicaffeate; 13 is betulin-3-caffeate; 14 is betulin-28-caffeate; and 15 is betulin 3,28,30-tricaffeate.

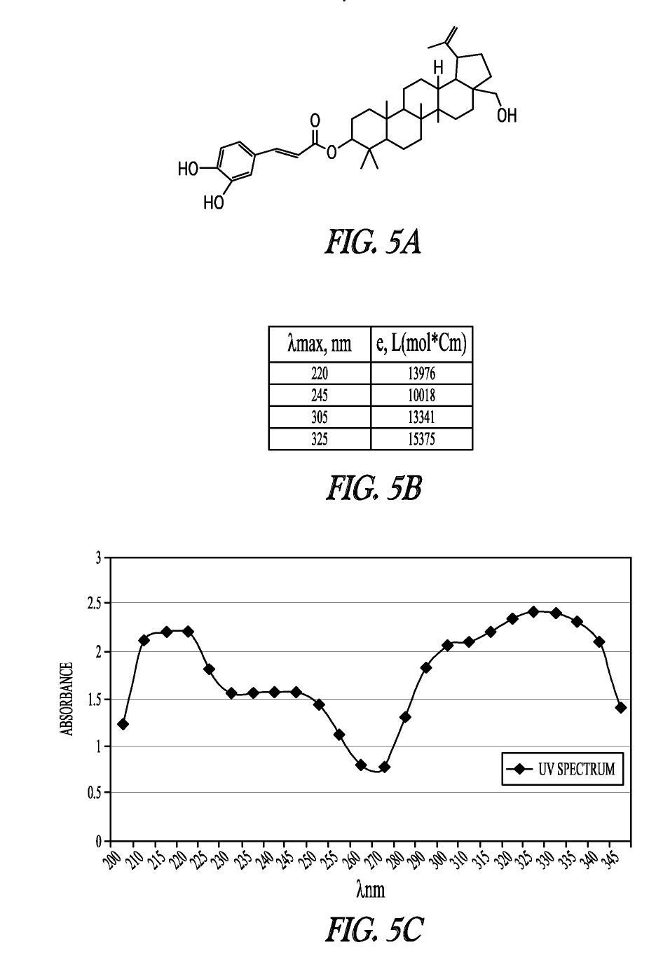

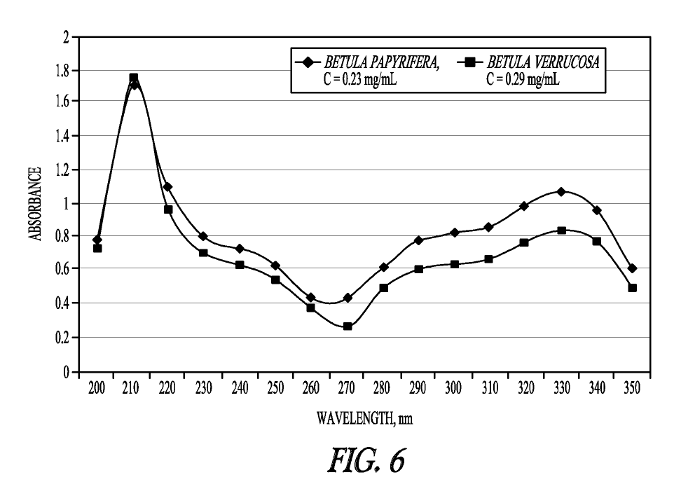

The analysis of birch bark extracts revealed useful characteristics of birch bark extract from outer bark of Betula papyrifera (paper birch) compared to the European birch bark of Betula pendula (Betula alba, Betula verucosa). For example, there was nearly 12 times higher concentration of betulin-3-caffeate in Betula papyrifera extract (6%), than in the European birch bark of Betula pendula (Betula alba, Betula verucosa)-0.5% (Ekman, R. and Sjoholm, R. “Betulinol 3-caffeate in outer bark of Betula verrucosa Ehrh.” Finnish Chemical Letters (1983) 134-6.). This characteristic (a natural bearer of high concentration of betulin-3-caffeate) means that birch bark extract from Betula papyrifera will be nearly 12 times better than extract from European birch bark, Betula pendula, as sun block material for skin protection (see, for example, FIGS. 5-6) or as an anti-oxidant, anti-cellulite, anti-cancer, anti-bacterial or anti-fungal (including toenail fungus) agent.

A hazard of prolonged exposure to sunlight is erythema (i.e., sunburn). The 290 to 320 nanometer wavelength ultraviolet radiation range, which is designated by the cosmetic industry as being the “UVB” wavelength rang, is the most effective type of UV radiation for producing erythema. The 320 to 400 nanometer wavelength ultraviolet radiation range, which is designated by the cosmetic industry as being the “UVA” wavelength range, also produces erythema. Thus, as can been seen from the UV absorbance curves (see FIGS. 5-6), the extracts, as well as betulin 3-caffeate, provide skin protection against sunburn.

### Example 3

### Effect of Betula Extract Components on Cell Growth Inhibition

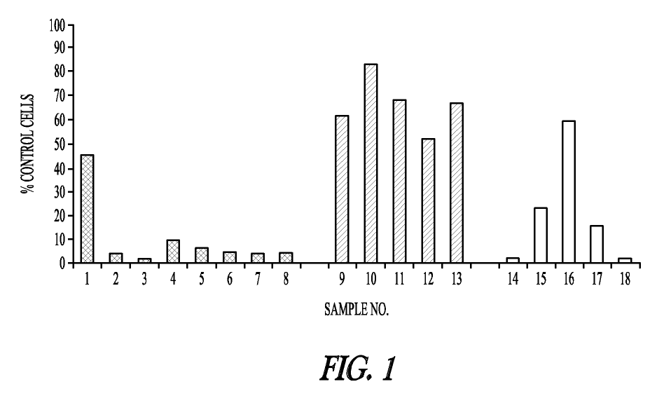

A single dose of 20 μg/ml of either extract, pure compound, or a mixture of compounds, was added to cultures of P19 stem cells, and the growth of the cells measured after 48 hours of treatment (FIG. 1). The extracts significantly inhibited cell proliferation, with the Alaskan species (B. neoalaskana and B. kenaica) appearing to be somewhat more effective than the Minnesota species (B. papyrifera). Pure betulinic acid and betulin-3-caffeate even more profoundly inhibited cell growth, but oleanic acid, lupeol, betulin and a mixture of suberinic acid and betulinic acid were less effective. These results suggest that betulinic acid and betulin-3-caffeate are the most active components in cell growth inhibition.

### Example 4

### Dose Response Studies

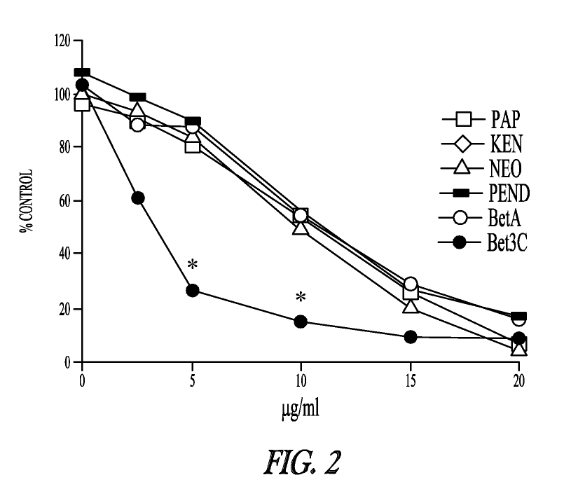

Dose response studies demonstrate that betulin-3-caffeate more effectively inhibits P19 stem cell growth that betulinic acid, which shows a similar level of effect as the extracts (FIG. 2). At concentrations of 5-10 μg/ml, betulin-3-caffeate is about 4 to 5 fold more potent than betulinic acid in inhibiting growth of P19 cells. Betulin 3-caffeate is significantly more effective than the other preparations in inhibiting cell growth (asterisks, p<0.05).

### Example 5

### Effect on Different Cancer Lines

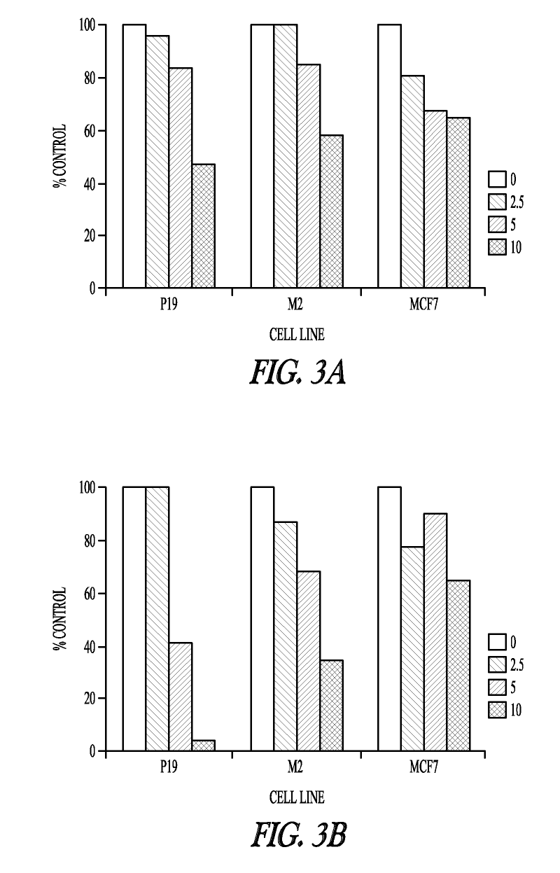

Comparing the responses of different cancer cell lines indicates that betulin-3-caffeate more effectively inhibits the growth of other types of malignant cells, including melanoma cells (FIG. 3). Overall, these results suggest that betulin-3-caffeate is the most active principal component in Betula extracts, and is more potent than betulinic acid in inhibiting the growth of a number of different types of cancer cells. Like the P19 cells, M2 melanoma cells are significantly inhibited by these triterpenoids, but the MCF-7 breast cancer cells do not appear to be as sensitive. In all cases, however, betulin-3-caffeate is more effective in inhibiting cell growth than betulinic acid.

### Example 6

### Response Studies with Different Preparations from the Extract

The survival of P19 stem cells after 48 h exposure to pure compounds and mixtures of Betula extracts is detailed in Table 2 and FIG. 1.

TABLE 2

| Sample | | | |
|---|---|---|---|
| No. | Chem. I.D. | Description | % Control |
| 1 | ie23n6s6 | mix of suberinic with betulinic acids | 45.2 |
| 2 | ie22n5s3 | Betula neoalaskana extract | 3.5 |
| 3 | ie22n6s5 | Betula kenaica extract | 1.4 |
| 4 | ie22n6s6 | Betula kenaica extract | 9.3 |
| 5 | ie22n6s7 | Betula papyrifera extract | 5.8 |
| 6 | ie22n6s4 | Betula kenaica extract | 4.1 |
| 7 | ie22n6s2 | Betula neoalaskana extract | 3.3 |
| 8 | ie22n6s1 | Betula neoalaskana extract | 3.6 |
| 9 | ie23n6s4 | Docosandioic acid | 61.5 |
| 10 | ie23n6s1 | Hydroxyoctadecanoic acid | 82.6 |
| 11 | ie23n6s2 | Treo-hydroxyoctadecanoic acid | 67.3 |
| 12 | ie23n6s10 | 22-hydroxydocosanoic acid | 51.4 |
| 13 | ie23n6s8 | Cis-18-hydroxyoctadec-9-enoic acid | 65.9 |
| 14 | ie24n6s2 | Betulinic acid | 1.3 |
| 15 | ie24n6s5 | Oleanolic acid | 22.3 |
| 16 | ie24n6s4 | Lupeol | 58.9 |
| 17 | ie24n6s3 | Betulin | 14.8 |
| 18 | ie24n6s1 | Caffeoxylupeol | 1.2 |

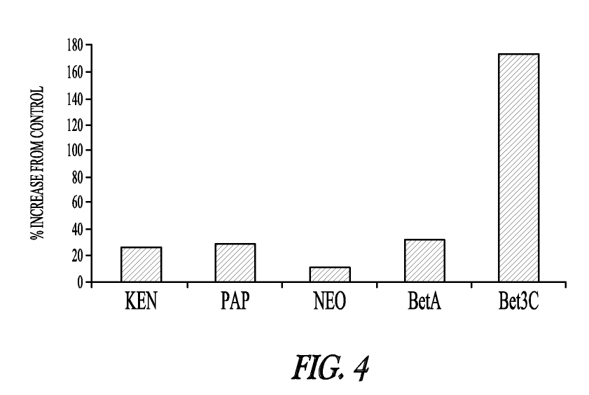

Stock solutions of compounds and extracts were diluted to 2 mg/ml in dimethylsulfoxide (DMSO), and subsequently added to samples at a final concentration of 20 μg/ml (10 μl/ml of a 2 mg/ml stock solution); controls received an equivalent amount of DMSO only (10 μl/ml). After 48 h, samples were fixed with methanol-acetic acid, and the quantity of surviving cells determined by sulforhodamine B staining methods (discussed above). The quantity of surviving cells is expressed as a percentage of the control (DMSO-only) cultures, which was set to 100%. Note that betulinic acid (sample 14) and betulin 3-caffeate (sample 18) are among the most active compounds in inhibiting cell proliferation. FIG. 4 shows the survival of P19 cells at 48 hours with respect to selected extract components. Extracts and betulinic acid induce a relatively modest increase in the numbers of dead cells present the cultures (about 10-40%). However, betulin 3-caffeate resulted in close to a 200% increase in the quantity of dead cells present, indicating that this triterpenoid is able to trigger cell death in malignant cells.

### Example 7

### Synthesis of Betulin-3-caffeate

### Synthetic Scheme I

The isolation of betulin-3-caffeate from Betula papyrifera was confirmed by chemical synthesis. In the course of this work, a novel method for preparation of betulin-3-caffeate from natural betulin was devised.

In a preferred embodiment of a method according to the present invention, betulin 1 and a haloacetyl halide are brought into contact in a dipolar aprotic solvent. Preferably the haloacetyl halide is bromoacetyl bromide, and the dipolar aprotic solvent is N,N-dimethylacetamide. The reactants may be brought into contact for any suitable time and at any suitable temperature at which the reaction proceeds to completion to yield the 3-O,28-O-bis(bromoacetyl)betulin 2, but preferably a temperature of about 50° C. and a time of about four hours at that temperature are employed. The product is purified by partitioning the reaction mixture between benzene and water, then washing the organic phase with additional water to remove the water-soluble N,N-dimethylacetamide.

The 3-O,28-O-bis(bromoacetyl)betulin 2 is selectively hydrolyzed to provide 3-O-bromoacetylbetulin 3. Preferably, the 3,28-bis(bromoacetyl)betulin is contacted with a solution of aluminum isopropoxide in isopropanol to cleave the ester group bonded to the primary C-28 hydroxyl group while leaving the secondary C-3 hydroxyl group in its esterified form. About two molar equivalents of aluminum isopropoxide are used. The reaction may be carried at any suitable temperature and for any suitable period of time, but preferably the reagents are in contact for about 78 minutes at a temperature of about 61° C.

The 3-bromoacetylbetulin 3 is then contacted with triphenylphosphine to provide 3-O-triphenylphosphoniumacetylbetulin bromide 4. Preferably the reagents are contacted in benzene solution for about 24 hours at ambient temperature. The phosphonium salt 4 may be isolated by any suitable means, but preferably it is isolated by dissolving in dichloromethane and precipitating with diethyl ether.

The phosphonium salt 4 is then coupled with 3,4-dihydroxybenzaldehyde in the presence of base to provide betulin-3-caffeate 5. Preferably the base is solid potassium bicarbonate, and the contacting is carried out in a solvent mixture of chloroform and dioxane. Unreacted 3,4-dihydroxybenzaldehyde is removed as its bisulfite addition compound by water extraction. The crude product may be purified by any suitable means, but preferably by column chromatography on silica gel to provide betulin 3-caffeate that was found to be identical with betulin 3-caffeate isolated from birch bark and with betulin 3-caffeate prepared by condensation of betulin and caffeic acid.

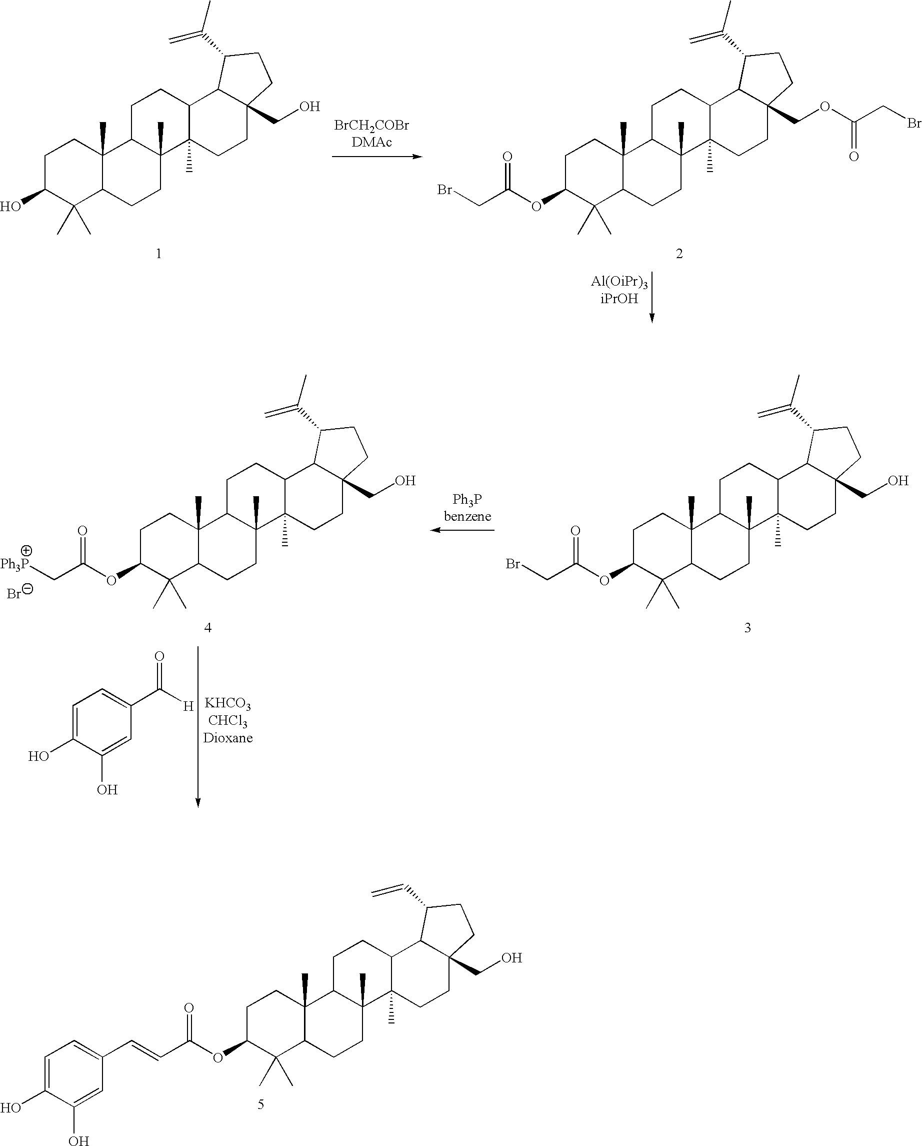

### (3β)-3,28-Di(bromoacetoxy)lup-20(29)-ene (2)

Betulin 1 (30.0 g, 0.0678 mole) was dissolved in dimethylacetamide (200 mL) and bromoacetyl bromide (25 mL) was added. The reaction mixture was stirred at 50° C. for 4 hrs and then kept at room temperature overnight. The mixture was diluted with benzene (300 mL) and the organic phase washed with water to remove dimethylacetamide. The organic phase was dried over Na₂SO₄ and the solvent evaporated. The solid residue was crystallized from isopropanol (300 mL) to yield compound 2 (34 g, 74%).

¹H NMR (CDCl₃+(CD₃)₂SO, 300 MHz): δ 4.74 (s, 1H), 4.58 (s, 1H), 4.46 (m, 1H), 4.38 (d, J=6 Hz, 1H), 4.22-4.08 (m, 4H), 2.5 (m, 1H), 1.83-0.60 (m, 45H).

¹³C NMR (CDCl₃+(CD₃)₂SO, 75 MHz): δ 167.64, 167.04, 149.87, 110.03, 83.186, 64.74, 55.34, 50.21, 48.79, 47.67, 46.55, 42.69, 40.86, 38.27, 38.05, 37.60, 37.02, 34.41, 34.05, 29.59, 29.49, 27.88, 26.99, 26.36, 25.99, 25.09, 23.44, 20.77, 19.10, 18.07, 16.40, 16.127, 15.99, 14.74.

### (3β)-3-Bromoacetoxylup-20(29)-en-28-ol (3)

(3β)-3,28-Di(bromoacetoxy)lup-20(29)-ene (2) (15 g, 21.9 mmol) was dissolved in dry i—PrOH (200 mL). The resulting solution was added to a solution of Al(O-i—Pr)₃ (8.92 g, 43.75 mmol) in dry i—PrOH (175 mL) at 61° C. The reaction mixture was stirred for 78 min at 61° C. The reaction was followed by HPLC.* The reaction mixture was quenched with a 5% solution of HCl in ice-water (1 L), and extracted with CH₂Cl₂ (5×50 mL). The combined organic extract was washed with H₂O and dried over Na₂SO₄. After solvent evaporation the residue was crystallized from i—PrOH (300 mL) at 5° C., yielding (3β)-3-bromoacetoxylup-20(29)-en-28-ol (9.6 g, 78%).

¹H NMR (CDCl₃, 300 MHz): δ 4.68 (s, 1H), 4.58 (s, 1H), 4.51 (dd, J₁=10.2 Hz, J₂=6.6 Hz, 1H), 3.85 (d, J=12 Hz, 1H), 3.78 (m, 2H), 3.32 (d, J=10.8 Hz, 1H), 2.41 (td, J₁=9.5 Hz, J₂=5.6 Hz, 1H), 1.98 (m, 3H), 1.9-0.75 (m, 40H).

¹³C NMR APT (CDCl₃, 75 MHz, δ_{CDCl3}=77.0): δ 167.0 (+), 150.4 (+), 109.7 (+), 83.2 (−), 60.5 (+), 55.3 (−), 50.2 (−), 48.7 (−), 47.8 (−), 47.7 (+), 42.7 (+), 40.9 (+), 38.3 (+), 38.0 (+), 37.2 (−), 37.0 (+), 34.0 (+), 33.9 (+), 29.6 (+), 29.1 (+), 27.9 (−), 26.9 (+), 26.3 (+), 25.1 (+), 23.4 (+), 20.8 (+), 19.0 (−), 18.1 (+), 16.4 (−), 16.1 (−), 15.9 (−), 14.7 (−).

IR (KBr): 3610 (bs), 2980, 1748, 1252 cm⁻¹. Anal. Calc'd for C₃₂H₅₁BrO₃: C, 68.19; H, 9.12. Found: C, 68.02; H, 9.01.

* HPLC conditions:

The analyses were performed on a Shimadzu (Shimadzu Scientific Instruments, Inc., Columbia, Md., U.S.A.) liquid chromatographic system consisting of a Model SCL 10 Avp system controller, a Model DGU-14A on-line degasser, a Model LC-10A Tvp HPLC pump, a Model FCV-10 ALvp low-pressure gradient flow control valve, a Model 7725i injector with 20 μL loop, and a Model SPD-10Avp diode array detector. The detector parameters were as follows: scan range 190-400 nm; 3-bromoacetoxylup-20(29)-en-28-ol was determined at 200 nm. For data acquisition and analysis the Shimadzu EZStart Ver. 7.2.SP1 was used. The chromatographic column used was a Discovery™ C18 reverse phase column, 5μ particle size, 250×4.6 mm I.D., (Supelco Inc. Catalog #504971). Elution was carried out in the isocratic mode at a flow rate of 0.5 mL/min. using an acetonitrile (100%) mobile phase.

### (3β)-3-(Triphenylphosphonium)acetoxylup-20(29)-en-28-ol Bromide (4)

Ph₃P (4.6 g, 17.6 mmol) was added to a solution of 3-bromoacetoxylup-20(29)-en-28-ol (3) (9.5 g, 16.9 mmol) in benzene (230 mL) and stirred at room temperature for 24 hrs. The solvent was evaporated and the residue was washed with Et₂O (20 mL), dissolved in CH₂Cl₂ (60 mL) and Et₂O (60 mL) was added dropwise until all 4 was precipitated. After filtration, the solid portion was re-precipitated three times from CH₂Cl₂/Et₂O. After drying at 50° C., (3β)-3-(triphenylphosphonium)acetoxylup-20(29)-en-28-ol bromide 4 (9.3 g, 67%) was obtained. m.p. 181.5-183° C.

¹H NMR (CDCl₃, 300 MHz): δ 7.95 (m, 6H), 7.89 (m, 3H), 7.67 (m, 6H), 5.75 (d, J=14.7 Hz, 0.5H), 5.69 (d, J=14.7 Hz, 0.5H), 5.40 (d, J=14.7 Hz, 0.5H), 5.34 (d, J=14.7 Hz, 0.5H), 4.67 (s, 1H), 4.57 (s, 1H), 4.33 (dd, J₁=10.5 Hz, J₂=6.3 Hz, 1H), 3.75 (d, J=10.8 Hz, 1H), 3.28 (d, J=10.8 Hz, 1H), 2.39 (td, J₁=9.5 Hz, J₂=5.6 Hz, 1H), 1.97 (m, 3H), 1.90-0.75 (m, 40H).

¹³C NMR APT (CDCl₃, 75 MHz, δ_{CDCl3}=77.0): δ 164.4 (+), 164.3 (+), 150.4 (+), 135.0 (−), 134.1 (−), 133.9 (−), 130.3 (−), 130.1 (−), 118.6 (+), 117.4 (+), 109.6 (+), 84.8 (−), 60.2 (+), 55.2 (−), 50.1 (−), 48.6 (−), 47.7 (−), 47.6 (+), 42.6 (+), 40.8 (+), 38.2 (+), 37.6 (+), 37.1 (−), 36.8 (+), 33.9 (+), 29.7 (+), 29.0 (+), 27.8 (−), 27.7 (−), 26.9 (+), 24.9 (+), 23.2 (+), 20.7 (+), 19.0 (−), 17.9 (+), 16.4 (−), 16.3 (−), 16.1 (−), 16.0 (−), 15.9 (−), 15.8 (−), 14.6 (−), 14.5 (−). Anal. Calc'd for C₅₀H₆₆BrO₃P: C, 72.71; H, 8.05; Br, 9.67. Found: C, 72.69; H, 7.99; Br, 9.64.

### (3β)-3-Caffeyloxylup-20(29)-en-28-ol (5)

3,4-Dihydroxybenzaldehyde (0.25 g, 1.82 mmol) was added to a solution of (3 β)-3-(triphenylphosphonium)acetoxylup-20(29)-ene-28-ol bromide 4 (1.5 g, 1.82 mmol) in freshly distilled dry dioxane (12 mL) and CHCl₃ (12 mL). Then, KHCO₃ (0.9 g, 9 mmol) was added to the solution and the reaction mixture was stirred for 24 hrs at 60° C. The solution was filtered and solvent was evaporated at 50° C. The residue was purified by column chromatography on silica gel using a mixture of diethyl ether/hexanes (1:4) as the eluting solvent. Then, changing the eluting solvent to diethyl ether/hexanes (1:1), crude 5 was obtained. Crude 5 was dissolved in diethyl ether (15 mL) and a saturated solution of NaHSO₃ in water was added and stirred for 1 hrs at room temperature. The organic layer was separated, washed with water (2 times), and dried over Na₂SO₄. After solvent evaporation (3β)-3-caffeyloxylup-20(29)-en-28-ol 5 (0.269 g, 25%) was obtained.

¹H NMR (CDCl₃, 300 MHz): δ 7.56 (d, J=15.9 Hz, 1H), 7.11 (d, J=1.8 Hz, 1H), 7.03 (dd, J₁=1.8 Hz, J₂=8.4 Hz, 1H), 6.88 (d, J=8.4 Hz, 1H), 6.28 (d, J=15.9 Hz, 1H), 5.82 (m, 2H, OH), 4.70 (s, 1H), 4.61 (m, 2H), 3.82 (d, J=10.8 Hz, 1H), 3.35 (d, J=10.8 Hz, 1H), 2.39 (td, J₁=10.5 Hz, J₂=5.6 Hz, 1H), 1.98 (m, 3H), 1.90-0.75 (m, 40H). Literature spectroscopic data are with agreement with our data for betulin 3-caffeate ((3β)-3-Caffeyloxylup-20(29)-en-28-ol 5). Anal. Calc'd for C₃₉H₅₆O₅: C, 77.44; H, 9.33.

Found: C, 77.40; H, 9.22. The structure was further verified by comparison with an authentic sample of betulin 3-caffeate isolated from birch bark.

### (3β)-3-Caffeyloxylup-20(29)-en-28-ol (5) by Extraction from Birch Bark

An isopropanol extract of outer birch bark (100 g) was dissolved in tetrahydrofuran (1 L) at 60° C. The mixture was cooled to room temperature, and aluminum isopropoxide (10 g, 49 mmol) was added, and the mixture stirred for 2 hrs. Water (1.7 g, 94.4 mmol) was then added. The resulting precipitate was filtered out and the solid washed with tetrahydrofuran (200 mL), then dried. The precipitate was washed with a 10% solution of acetic acid in water, dried, then extracted with a 1% solution of acetic acid in isopropyl alcohol (300 mL). The combined extracts were concentrated by solvent evaporation and the crude material was purified by flash chromatography on silica gel using diethyl ether as the eluent. The combined fractions were evaporated and further purified by column chromatography on silica gel using diethyl ether/hexanes 2:1 as the eluent. Fractions containing betulin 3-caffeate were combined and solvent was evaporated to give 4.2 g (4.2%) of crystals. m.p. 191.1-198.3° C.

¹H NMR (CDCl₃, 300 MHz): δ 7.56 (d, J=15.9 Hz, 1H), 7.11 (d, J=1.8 Hz, 1H), 7.03 (dd, J₁=1.8 Hz, J₂=8.4 Hz, 1H), 6.88 (d, J=8.4 Hz, 1H), 6.28 (d, J=15.9 Hz, 1H), 5.82 (m, 2H, OH), 4.70 (s, 1H), 4.61 (m, 2H), 3.82 (d, J=10.8 Hz, 1H), 3.35 (d, J=10.8 Hz, 1H), 2.39 (td, J₁=10.5 Hz, J₂=5.6 Hz, 1H), 1.98 (m, 3H), 1.90-0.75 (m, 40H). Literature spectroscopic data are in agreement with our data for betulin 3-caffeate ((3β)-3-Caffeyloxylup-20(29)-en-28-ol (5)). Anal. Calc'd for C₃₉H₅₆O₅: C, 77.44; H, 9.33. Found: C, 77.40; H, 9.22.

### Example 8

### Synthesis of Mono-Silylated Betulin

### Synthetic Scheme II

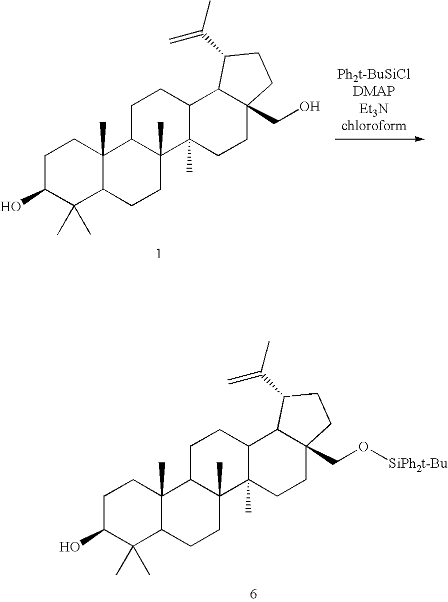

### (3β)-28-t-Butyldiphenylsilyloxylup-20(29)-en-3-ol (6)

DMAP (1.51 g, 12.4 mmol) and Et₃N (1.25 g, 12.5 mmol) were added to a solution of betulin (1 g, 2.26 mmol) in dry CHCl₃ (50 mL). Tert-Butyldiphenylchlorosilane (3.1 g, 11.3 mmol) was then added to the reaction mixture. The mixture was refluxed for 36 hrs, then was cooled down and the solution washed with water (2×15 mL), then with a 5% solution of HCl in water (5×10 ml), and then with a saturated solution of NaCl, then was dried over Na₂SO₄. NMR analysis of the reaction mixture showed no presence of betulin or of the bis-(3,28)-silyl derivative of betulin. The residue remaining after CHCl₃ evaporation was purified by column chromatography on silica gel with diethyl ether/hexane 1:1 as the eluent. Fractions containing 6 were combined and the solvent was evaporated to give 1.39 g (90%) of (3β)-28-t-butyldiphenylsilyloxylup-20(29)-en-3-ol 6.

¹H NMR (CDCl₃, 300 MHz): δ 7.68 (m, 4H), 7.11 (m, 6H), 4.59 (s, 1H), 4.52 (s, 1H), 3.68 (d, J=9.9 Hz, 1H), 3.32 (d, J=9.9 Hz, 1H), 3.16 (dd, J₁=10.8 Hz, J₂=5.8 Hz, 1H), 2.39 (td, J₁=10.5 Hz, J₂=5.4 Hz, 1H), 2.13 (m, 2H), 1.95 (m, 1H), 1.90-0.75 (m, 49H).

¹³C NMR APT (CDCl3, 75 MHz, δ_{CDCl3}=77.0): δ 150.8 (+), 135.6 (−), 133.9 (+), 133.9 (+), 129.5 (−), 127.6 (−), 109.4 (+), 78.9 (−), 61.0 (+), 50.3 (−), 48.5 (−), 48.4 (−), 47.8 (+), 42.6 (+), 40.7 (+), 38.8 (+), 38.6 (+), 37.2 (−), 37.0 (+), 34.5 (+), 34.1 (+), 29.8 (+), 29.5 (+), 27.9 (−), 27.6 (−), 27.3 (+), 27.0 (+), 26.9 (−), 26.2 (−), 25.1 (+), 20.7 (+), 19.4 (+), 19.1 (−), 18.3 (+), 16.1 (−), 16.0 (−), 15.7 (−), 15.6 (−), 15.4 (−), 15.3 (−), 14.7 (−).

Anal. Calc'd for C₄₈H₆₈O₂Si: C, 81.12; H, 10.06. Found: C, 81.02; H, 10.01.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A composition comprising at least two of:
(a) betulin 3-caffeate; (b) betulinic acid; (c) oleanolic acid; (d) betulin; (e) lupeol; (f) 3-acetoxyoleanolic acid; (g) betulin aldehyde; (h) betulonic aldehyde; and (i) pycarehic acid (betulinic acid-3-caffeate);
wherein the composition is essentially free of plant tissue.

2. The composition of claim 1, wherein the betulin 3-caffeate is present at a concentration of greater than or equal to about 5 μM.

3. The composition of claim 1, wherein the betulin 3-caffeate is present in amounts up to about 30.0 wt. % of the composition.

4. The composition of claim 1, wherein the betulinic acid is present in amounts up to about 30.0 wt. % of the composition; the oleanolic acid is present in amounts up to about 30.0 wt. % of the composition; the betulin is present in amounts up to about 80.0 wt. % of the composition; the lupeol is present in amounts up to about 20.0 wt. % of the composition; the 3-acetoxyoleanolic acid is present in amounts up to about 30.0 wt. % of the composition; the betulin aldehyde is present in amounts up to about 10.0 wt. % of the composition; and the betulonic aldehyde is present in amounts up to about 1.0 wt. % of the composition.

5. The composition of claim 1, further comprising one or more of ursolic acid, moronic acid; platonic acid; papyriferic acid; ursolic acid caffeate; or oleanolic acid caffeate.

6. The composition of claim 1, which is in the form of a cream, lotion, gel, ointment, emollient, powder, eye drop, liposome, tablet, capsule, liquid or solid.

7. The composition of claim 1, which is admixed with a food product, food beverage, sport drink, health bar, a dietary supplement, a vitamin supplement, a mineral supplement, a food oil, or a combination thereof.

8. The composition of claim 1, which is derived from one or more plant tissues of birch bark, olive skin, cranberry skin, apple skin, clove tree seed, clove tree bark, clove tree root, sycamore tree bark, sycamore tree root, grape skin, or blueberry skin.

9. The composition of claim 1, further comprising a plant stanol, a plant stanol ester, a plant stanol ester that includes less than about 1 wt. % cholesterol, one or more natural saturated oils, one or more unnatural saturated oils, or at least one of a pharmaceutically acceptable carrier, diluent, solvent, filler, lubricant, excipient, binder and stabilizer, or a combination thereof.

10. The composition of claim 1, wherein the composition comprises less than about 10.0 wt. % plant tissue.

11. A composition comprising:
(a) betulin 3-caffeate; (b) betulinic acid; (c) oleanolic acid; (d) betulin; (e) lupeol; (f) 3-acetoxyoleanolic acid; (g) betulin aldehyde; (h) betulonic aldehyde; and (i) pycarehic acid (betulinic acid 3-caffeate);
wherein the composition is essentially free of plant tissue.

12. The composition of claim 11, comprising (a) up to about 10.0 wt. % of betulin 3-caffeate; (b) up to about 20.0 wt % of betulinic acid; (c) up to about 10.0 wt. % of oleanolic acid; (d) up to about 80.0 wt. % of betulin; (e) up to about 15.0 wt. % of lupeol; (f) up to about 15.0 wt. % of 3-acetoxyoleanolic acid; (g) up to about 1.5 wt. % of betulin aldehyde; (h) up to about 1.0 wt. % of betulonic aldehyde; and (i) up to about 10.0 of pycarehic acid (betulinic acid 3-caffeate); wherein the composition is essentially free of plant tissue.

13. A method of treating a hyperproliferative disease in a mammal, comprising administering to the mammal in need of such treatment the composition of claim 1 in a dosage, at a frequency, and for a duration of time sufficient to provide a beneficial result.

14. A method of treating a hyperproliferative disease in a mammal, comprising administering to the mammal in need of such treatment an effective amount of a compound of formula (I): 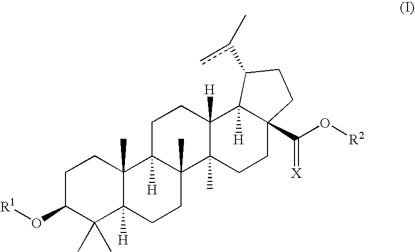
or a pharmaceutically acceptable salt thereof, wherein,
R¹ is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl;
R² is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl;
X is absent, O or S; and
the bond represented by [symbol] is absent or present;
or an effective amount of a compound of formula (II): 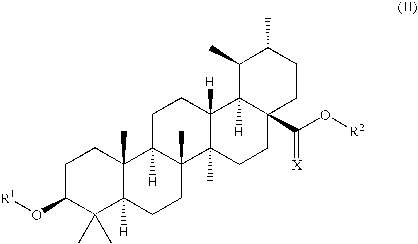
or a pharmaceutically acceptable salt thereof, wherein,
R¹ is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl;
R² is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl; and
X is absent, O or S;
or an effective amount of a compound of formula (III): 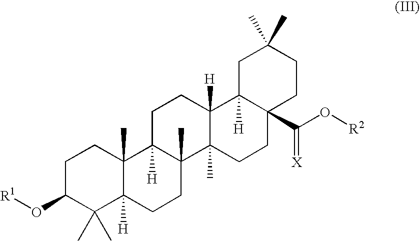
or a pharmaceutically acceptable salt thereof, wherein,
R¹ is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl;
R² is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl; and
X is absent, O or S;
in a dosage, at a frequency, and for a duration of time sufficient to provide a beneficial result.

15. The method of claim 14 wherein R¹ and R² are each independently H or —R³R⁴R⁵, wherein R³ is oxo (═O), thioxo (═S), CH(OH), or NR⁶, wherein R⁶ is H, alkyl, cycloalkyl, or aryl; R⁴ is alkyl or alkenyl; and R⁵ is cycloalkyl, heterocyclyl, aryl, or heteroaryl.

16. The method of claim 14 wherein R¹ is a substituent of the formula (IV): 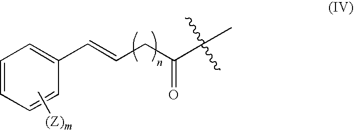
wherein,
the non-aromatic carbon-carbon double bond is in the cis- or trans-configuration;
n is 0-5; m is 0-5; each Z is independently H, OH or hydroxyalkyl; and the wavy line indicates a point of attachment.

17. A method selected from the group consisting of treating a hyperproliferative disease, providing an antibiotic treatment, providing a dietary supplement, providing a skin care supplement, treating a fungal or a bacterial infection or a protozoan or a parasitic infestation, in a mammal, in a mammal;
the method comprising administering a compound of formula (IVA) in a dosage, at a frequency, for a duration of time, and to a site on or within the mammal, sufficient to treat the mammal; 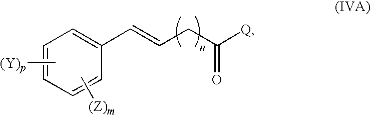
wherein
the non-aromatic carbon-carbon double bond is in the cis- or trans-configuration;
n is 0-5; m is 0-5;
p is 0-5, provided that m+p is less than or equal to a total of 5;
each Y is independently alkyl, alkenyl, alkoxy, halo, haloalkyl, hydroxy, hydroxyalkyl, aryl, heteroaryl, heterocycle, cycloalkyl, alkanoyl, acyloxy, alkoxycarbonyl, acylamino, nitro, trifluoromethyl, trifluoromethoxy, carboxy, carboxyalkyl, alkylthio, alkylsulfinyl, alkylsulfonyl, cyano, acetamido, acetoxy, acetyl, arylamido, arylsulfinyl, arylsulfonamido, arylsulfonyl, arylsulfonylamino, aroyl, arylamino, aroyloxy, aralkyl, aralkyloxy, aralkyloxycarbonyl, aralkylthio, carbamoyl, carbamate, isocyanato, sulfamoyl, sulfinamoyl, sulfino, sulfo, sulfoamino, thiosulfo, NR^{x}R^{y} or COOR^{x}, wherein each R^{x} and R^{y} is independently at each occurrence H, or substituted or unsubstituted alkyl, alkenyl, aryl, heteroaryl, heterocyclyl, or cycloalkyl; each Z is independently H, OH or hydroxyalkyl; and
Q comprises a residue of betulin, betulinic acid, ursolic acid, oleanic acid, allobetulin, allobetulin lactone, lupeol, or a pentacyclic triterpene alcohol; bonded by a hydroxyl thereof to the carbonyl group.

18. The method of claim 17, wherein the compound of formula (I) is betulin 3-caffeate.

19. The method of claim 17 wherein the hyperproliferative disease is non-malignant and primarily caused by overactive cell cycle activity, and wherein the hyperproliferative disease comprises atherosclerosis, rheumatoid arthritis, psoriasis, idiopathic pulmonary fibrosis, scleroderma or cirrhosis of the liver, the hyperproliferative disease is non-malignant and primarily caused by a reduced level of normal programmed cell death (apoptosis), and wherein the hyperproliferative disease comprises atherosclerosis, rheumatoid arthritis, psoriasis, idiopathic pulmonary fibrosis, scleroderma or cirrhosis of the liver, the hyperproliferative disease is caused by increased cell division (overactive cell cycle activity), reduced cell death (reduced apoptotic activity), reduced necrotic activity, reduced autophagy or a combination thereof, the hyperproliferative disease is pre-malignant or malignant and wherein the hyperproliferative disease comprises hyperplasias, metaplasias, nevi, leukemias, sarcomas, adenomas, carcinomas, gliomas, melanomas, and all other types of pre-neoplastic and neoplastic growth, whether resulting from overactive cell cycle activity, reduced apoptotic activity, reduced necrotic activity, reduced autophagy or a combination thereof, and wherein the pre-malignant and malignant diseases optionally comprise those arising from either transformation of differentiated cells or the transformation of stem cells, stem cell progeny, or hybrids of stem cells and differentiated cells.

20. A method of providing topical UV-protection to a mammal or treating cancer associated with UV radiation, the method comprising topically applying the composition of claim 1, or a compound of formula (I): 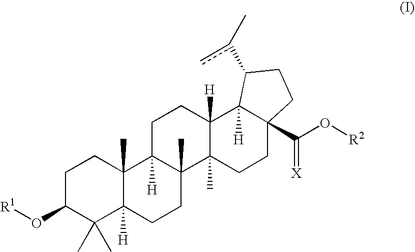
or a pharmaceutically acceptable salt thereof, wherein,
R¹ is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl;
R² is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl;
X is absent, O or S; and
the bond represented by [symbol] is absent or present;
or a compound of formula (II): 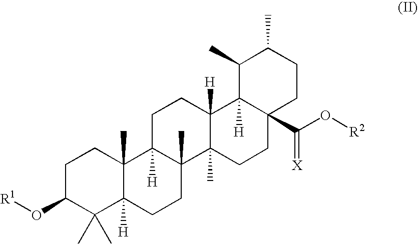
or a pharmaceutically acceptable salt thereof, wherein,
R¹ is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl;
R² is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl; and
X is absent, O or S;
or a compound of formula (III): 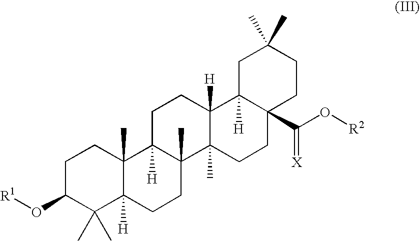
or a pharmaceutically acceptable salt thereof, wherein,
R¹ is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl;
R² is H, alkyl, alkenyl, haloalkyl, hydroxyalkyl, aryl, heteroaryl, heterocycle, cycloalkyl, alkanoyl, alkoxycarbonyl, carboxyalkyl, acetyl, benzoyl, benzyl, benzyloxycarbonyl, benzylthio, or carbamoyl; and
X is absent, O or S;
or a compound of formula (IVA): 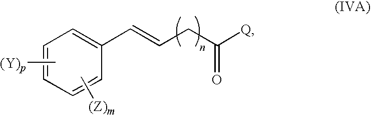
wherein
the non-aromatic carbon-carbon double bond is in the cis- or trans-configuration;
n is 0-5;
m is 0-5;
p is 0-5, provided that m+p is less than or equal to a total of 5;
each Y is independently alkyl, alkenyl, alkoxy, halo, haloalkyl, hydroxy, hydroxyalkyl, aryl, heteroaryl, heterocycle, cycloalkyl, alkanoyl, acyloxy, alkoxycarbonyl, acylamino, nitro, trifluoromethyl, trifluoromethoxy, carboxy, carboxyalkyl, alkylthio, alkylsulfinyl, alkylsulfonyl, cyano, acetamido, acetoxy, acetyl, arylamido, arylsulfinyl, arylsulfonamido, arylsulfonyl, arylsulfonylamino, aroyl, arylamino, aroyloxy, aralkyl, aralkyloxy, aralkyloxycarbonyl, aralkylthio, carbamoyl, carbamate, isocyanato, sulfamoyl, sulfinamoyl, sulfino, sulfo, sulfoamino, thiosulfo, NR^{x}R^{y} or COOR^{x}, wherein each R^{x} and R^{y} is independently at each occurrence H, or substituted or unsubstituted alkyl, alkenyl, aryl, heteroaryl, heterocyclyl, or cycloalkyl; each Z is independently H, OH or hydroxyalkyl; and
Q comprises a residue of betulin, betulinic acid, ursolic acid, oleanic acid, allobetulin, allobetulin lactone, lupeol, or a pentacyclic triterpene alcohol; bonded by a hydroxyl thereof to the carbonyl group;
to the mammal before the mammal is exposed to UV radiation.

21. A method of preparing a caffeate ester of an triterpenoid alcohol or a steroid alcohol, comprising:
contacting the alcohol with a compound of formula XCR¹R²C(O)Y, wherein X is a halogen and Y is an ester activation moiety selected from a group consisting of halides, N-hydroxy compounds, or phenols, R¹ and R² are each H to provide a haloacetyl ester; then,
contacting the haloacetyl ester of the alcohol with a triaryl phosphine to provide a triarylphosphoniumacetyl ester; and then,
contacting the triarylphosphoniumacetyl ester of the alcohol with 3,4-dihydroxybenzaldehyde in the presence of base, the base being carbonate or bicarbonate, to provide the caffeate ester of the alcohol.
